(19) United States
(12) Patent Application Publication
(10) Pub. No.: US 2024/0002917 A1
(43) Pub. Date: Jan. 4, 2024

(54) METHOD OF DETECTION OF A TARGET NUCLEIC ACID SEQUENCE

(71) Applicant: RARITY BIOSCIENCE AB, Uppsala (SE)

(72) Inventors: Ulf LANDEGREN, Uppsala (SE); Lei CHEN, Uppsala (SE)

(21) Appl. No.: 18/253,658

(22) PCT Filed: Dec. 2, 2021
(86) PCT No.: PCT/EP2021/084061

§ 371 Date: May 19, 2023

(30) Foreign Application Priority Data
Dec. 3, 2020 (GB) 2019074.0

(51) Int. Cl. C12Q 1/6844

(52) U.S. Cl. CPC C12Q 1/6844

(57) ABSTRACT

The present disclosure and invention relates to a method for detecting a target nucleic acid sequence in a target molecule using padlock probes and rolling circle amplification (RCA) in a 2-stage RCA reaction, a so-called superRCA (sRCA), also termed “SafeLock” herein, which generates a second-generation RCA product, by means of which the target nucleic acid sequence may be detected and distinguished from other nucleic acid sequences. The method relies on gap-fill-ligation padlock probe technology, and may be used to detect variant sequences that may occur in samples. Also provided are kits for use in the method.

### FIELD

The present disclosure and invention concerns the field of nucleic acid detection. Particularly, the present disclosure and invention relates to a method for detecting a target nucleic acid sequence in a target molecule using padlock probes and rolling circle amplification (RCA) in a 2-stage RCA reaction, a so-called superRCA (sRCA), also termed “SafeLock” herein, which generates a second-generation RCA product, by means of which the target nucleic acid sequence may be detected and distinguished from other nucleic acid sequences. The method relies on gap-fill-ligation padlock probe technology, and may be used to detect variant sequences that may occur in samples. Also provided are kits for use in the method.

### BACKGROUND

The detection of target nucleic acid sequences has applications in many different fields, including notably clinically, for personalised medicine and in the diagnosis, prognosis and/or treatment of disease, such as cancer, infectious diseases and inherited or genetic disorders, as well as in research and biosecurity.

Target nucleic acid sequence may readily be detected using labelled hybridisation probes, but simply hybridisation probes have relatively high lower detection limits, and cannot readily be used to discriminate between similar nucleic acid sequences. To increase sensitivity, target nucleic acid molecules containing target sequences may typically amplified, to increase the amount of target sequence available for detection. Any of a variety of techniques known in the art may be used for the amplification, including RCA.

RCA utilises a strand displacement polymerase enzyme, and requires a circular amplification template. Amplification of the circular template provides a concatenated RCA product, comprising multiple copies of a sequence complementary to that of the amplification template. Such a concatemer typically forms a ball or “blob”, which may readily be visualised and detected, and thus RCA-based assays have been adopted for the detection of nucleic acids, and indeed, more generally, as reporter systems for the detection of any target analyte. Both target nucleic acids, which may be themselves be circularised directly, or probes, or reporter nucleic acids more generally may provide template nucleic acid circles for RCA.

The specificity of a nucleic acid detection method may be improved by the use of probes which require dual recognition, or two binding sites for a target nucleic acid sequence, such as a padlock probe. Padlock probes are linear oligonucleotides with two separate target-complementary binding regions, connected by an intervening “backbone” region. When the probe has bound (hybridised) to its target nucleic acid sequence, the ends of the probe may be ligated together to circularise the probe. The circularised padlock probe may then be used as the template for a RCA reaction, and the RCA product may be detected. This forms the basis of a number of detection assays in use today. Padlock probes thus provide an extra layer of specificity, since only probes correctly base-paired at the ligation site will be ligated to generate the template for the molecule which will be detected. When the padlock probe hybridises to the target nucleic acid sequence with its target-binding sites directly adjacent to one another, the ends of the padlock probe may be ligated to each other directly. Alternatively the target-binding sites of the padlock probe may hybridise to the target nucleic acid with a gap in between, and the gap may be filled in, either by hybridisation of one or more gap oligonucleotides in the gap region, or by a polymerase-catalysed extension of the hybridised 3′ end of the probe. In this way, the hybridised ends of the padlock probe may ligated to each other indirectly, in that they are each hybridised to an intervening “gap sequence”. Such “gap fill” padlock probes, also known as molecular inversion probes, are described in Hiatt et al., 2013, Genome Res, 23, 843-854, wherein molecular inversion probes are used for targeted, high-accuracy detection of low-frequency variation.

RCA-based assays have been described which rely on secondary amplification of the initial RCA product, to increase the amount of product which is detected, and thereby to provide amplification of the signal in the assay. These include for example hyberbranched RCA. More recently so-called “super RCA (sRCA) reactions have been developed which comprise 2 or more rounds of RCA amplification, wherein the product of the second RCA reaction is linked to that of the first. Such a sRCA method is described in WO2014/0796209. In this method a probe capable of providing or functioning as a primer is hybridised to an initial RCA product and is used to prime the amplification of a second RCA template circle which hybridises to the “primer-probe”. The second RCA template may be generated by circularisation of a padlock probe which hybridises to the “primer-probe”. In WO 2015/071445 an alternative sRCA method, termed “Padlock sRCA” is described, in which a padlock probe is used to bind directly to the initial RCA product.

In many applications, the nucleic acid sequences to be detected occur at low levels, for example in the case of rare mutations, or cell-free DNA (cfDNA) in plasma or other clinical samples, or where limited amounts of sample are available. In such cases, very sensitive methods of detection are required, and particularly methods which allow a high amplification of the target nucleic acid to be achieved. Whilst the sRCA methods described above go some way towards providing improved and sensitive assays, there is a continuing need for highly sensitive nucleic acid detection methods. The present disclosure, and invention, are directed to providing such a method.

### SUMMARY

In the present method, a target-specific first padlock probe is used to generate a complementary copy of the target sequence by a gap-fill extension reaction. The circularised first padlock probe containing the complementary copy is then amplified by RCA to generate a first RCA product containing multiple copies of the target sequence. The resulting first RCA product is then probed with a further, second, padlock probe, specific for the target sequence. The circularised second padlock probe is subjected to a further, second RCA reaction, which is used to generate a second RCA product which is detected to detect the target sequence. As noted above, a 2-stage RCA reaction where a second padlock probe is used, which binds to a first RCA product, is referred to herein as a “SafeLock” assay, and accordingly, the second padlock may also be referred to as a “SafeLock padlock”.

The present method thus relies on at least two rounds of RCA amplification to generate the product to be detected. This in itself increases the amount of detectable product. However, by additionally designing the method so that the second padlock probe targets the sequence in the first RCA product that is the complement of the “gap-infill” sequence in the circularised first padlock probe (namely the sequence that is created by the gap-fill extension reaction to fill the gap in between the hybridised ends of the first padlock probe) the specificity of the method is improved. The second padlock probe is designed specifically to recognise the target sequence, multiple copies of which have been created in the first RCA product. Further, improvements have been made which increase the amount of RCA template that may be generated for the first RCA reaction. The improvements increase the efficiency of the gap-fill extension reaction, and allow more of the correctly-ligated circularised first padlock probe to be generated. In one embodiment, the improvements allow the probe binding, gap-filling, and ligation reactions to be combined in a single step, a so-called “gap-fill-ligation” step, wherein the padlock probe and the reagents for the gap-fill extension and ligation reactions are added together. This allows the gap-fill-ligation reactions to be cycled, thereby increasing the amount of circularised first padlock probe that is produced, and therefore increasing the efficiency and the sensitivity of the detection method. In another embodiment, the probe-binding step is separated from the gap-fill extension and ligation steps. The present method also provides a powerful means of screening DNA samples for the presence of a very large number of distinct target sequences.

Accordingly, in a first and broad aspect provided herein is a method for detecting a target nucleic acid sequence in a target nucleic acid molecule in a sample, said method comprising:
(i) contacting the target nucleic acid molecule with a first padlock probe, which comprises at or near its respective 5′ and 3′ ends target-binding regions which are capable of hybridising to complementary binding sites in the target nucleic acid molecule which flank the target nucleic acid sequence, and allowing the target-binding regions of the probe to hybridise to the target nucleic acid molecule; and
(ii) optionally after cleavage of any unhybridised nucleotides at the 5′ and/or 3′ ends, extending the hybridised 3′ end of the padlock probe using a polymerase to create a complementary copy of the target nucleic acid sequence, and ligating the extended 3′ end to the hybridised 5′ end to circularise the padlock probe;
(iii) performing a first RCA reaction using the circularised padlock probe as a first RCA template to generate a first RCA product (RCP) comprising multiple repeats of a copy of the target nucleic acid sequence;
(iv) contacting the first RCP with a second padlock probe comprising target binding regions specific for the target nucleic acid sequence and allowing the probe to hybridise to the target sequence in the multiple repeats;
(v) ligating the hybridised second padlock probe to circularise the hybridised padlock probe;
(vi) performing a second RCA reaction using the circularised second padlock probe as a second RCA template to generate a second RCP containing multiple repeat complementary copies of the second padlock probe;
(vii) detecting the second RCP to detect the second padlock probe, and thereby the target nucleic acid sequence.

In particular, in steps (ii) and (v) a ligase is used to ligate and circularise padlock probe.

In a more particular embodiment there is provided a method for detecting a target nucleic acid sequence in a target nucleic acid molecule in a sample, said method comprising:
(i) contacting the target nucleic acid molecule with a first padlock probe, which comprises at or near its respective 5′ and 3′ ends target-binding regions which are capable of hybridising to complementary binding sites in the target nucleic acid molecule which flank the target nucleic acid sequence, and allowing the target-binding regions of the probe to hybridise to the target nucleic acid molecule, wherein the 3′ target binding region of the padlock probe is at least 6 bases shorter than the 5′ target binding region and the padlock probe is contacted with the target nucleic acid molecule together with dNTPs, a polymerase and a ligase, and wherein the dNTPs are provided at a concentration of no more than 10 μM and the polymerase is provided at a concentration of no more than 0.025 U/uL;
(ii) optionally after cleavage of any unhybridised nucleotides at the 5′ and/or 3′ ends, extending the hybridised 3′ end of the padlock probe using the polymerase to create a complementary copy of the target nucleic acid sequence, and ligating the extended 3′ end to the hybridised 5′ end using the ligase to circularise the padlock probe;
(iii) performing a first RCA reaction using the circularised padlock probe as a first RCA template to generate a first RCA product (RCP) comprising multiple repeats of a copy of the target nucleic acid sequence;
(iv) contacting the first RCP with a second padlock probe comprising target binding regions specific for the target nucleic acid sequence and allowing the probe to hybridise to the target sequence in the multiple repeats;
(v) ligating the hybridised second padlock probe to circularise the hybridised padlock probe;
(vi) performing a second RCA reaction using the circularised second padlock probe as a second RCA template to generate a second RCP containing multiple repeat complementary copies of the second padlock probe;
(vii) detecting the second RCP to detect the second padlock probe, and thereby the target nucleic acid sequence.

In a further more particular embodiment, there is provided a method for detecting a target nucleic acid sequence in a target nucleic acid molecule in a sample, said method comprising:
(i) (a) contacting the target nucleic acid molecule with a first padlock probe, which comprises at or near its respective 5′ and 3′ ends target-binding regions which are capable of hybridising to complementary binding sites in the target nucleic acid molecule which flank the target nucleic acid sequence, and allowing the target-binding regions of the probe to hybridise to the target nucleic acid molecule; and
   (b) after the probe has hybridised to the target nucleic acid molecule, contacting the hybridised padlock probe/target nucleic acid reaction mixture with a polymerase;
(ii) optionally after cleavage of any unhybridised nucleotides at the 5′ and/or 3′ ends, extending the hybridised 3′ end of the padlock probe using the polymerase to create a complementary copy of the target nucleic acid sequence, and ligating the extended 3′ end to the hybridised 5′ end to circularise the padlock probe;
(iii) performing a first RCA reaction using the circularised padlock probe as a first RCA template to generate a first RCA product (RCP) comprising multiple repeats of a copy of the target nucleic acid sequence;
(iv) contacting the first RCP a second padlock probe comprising target binding regions specific for the target nucleic acid sequence and allowing the probe to hybridise to the target sequence in the multiple repeats;
(v) ligating the hybridised second padlock probe to circularise the hybridised padlock probe;
(vi) performing a second RCA reaction using the circularised second padlock probe as a second RCA template to generate a second RCP containing multiple repeat complementary copies of the second padlock probe;
(vii) detecting the second RCP to detect the second padlock probe, and thereby the target nucleic acid sequence.

In this method, by not providing the polymerase until after the first padlock probe has hybridised, the probe-binding step is separated from the gap-filling extension step, This method thus provides a 2-step protocol for probe binding and gap-fill extension. In such a method, dNTPs for the extension reaction, can be provided prior to, together with, or after the polymerase. A ligase for the ligation reaction can be provided prior to, with, or after the polymerase. There may be a washing after probe binding, e.g. before the polymerase is added, or before the extension reaction takes place. In particular, step i(b) takes place after the 5′ target binding region has hybridised to the target nucleic acid sequence.

The methods presented above may particularly be used to detect a variant target nucleic acid sequence in a target nucleic acid molecule in a sample. Target nucleic acid sequences may commonly occur in variant forms, for example allelic variants, or mutant and wild-type sequences, and it may be desirable to detect which variant is present. Accordingly, in an embodiment the target nucleic acid molecule is an analyte in a sample. However, in another embodiment, the target nucleic acid molecule is not itself the target analyte, but rather is detected as part of an assay to detect another target analyte. Accordingly in such an embodiment, the target nucleic acid molecule may be a reporter molecule for a target analyte.

In an embodiment, steps (iv) to (vii) of the methods above may comprise:
(iv) contacting the first RCP with two or more second padlock probes each comprising target binding regions specific for different variants of the target nucleic acid sequence and allowing the probes to hybridise to their respective variant target sequence in the multiple repeats, where it is present;
(v) ligating the second padlock probes which have hybridised to circularise the hybridised padlock probes;
(vi) performing second RCA reactions using the circularised padlock probes as a second RCA template to generate a second RCP containing multiple repeat complementary copies of the second padlock probe;
(vii) detecting the second RCP to identify the second padlock probe, and thereby the variant target nucleic acid sequence.

The method may be used for the detection of DNA or RNA, and may be performed in homogenous or heterogenous formats. It may be used to detect a target nucleic acid sequence in situ or in isolated form, or in a liquid sample.

Advantageously, the method may be performed in multiplex, to detect multiple different target nucleic acid sequences.

In an embodiment, the gap-fill extension and ligation steps are cycled to increase the amount of circularised first padlock probe which is generated.

The method may involve further cycles, or generations of RCA. Thus, steps (iv) to (vi) may be repeated, using a third padlock probe comprising target binding regions specific for the target nucleic acid sequence in the second RCP, ligating to circularise the third padlock probe, performing a third RCA reaction, and so on to generate 3^{rd}, 4^{th} or even further generation RCPs.

In a second aspect, there is provided a kit for use in detecting a target nucleic acid sequence in a target nucleic acid molecule, said kit comprising:
(i) a first padlock probe which comprises at or near its respective 5′ and 3′ ends target-binding regions which are capable of hybridising to complementary binding sites in the target nucleic acid molecule which flank the target nucleic acid sequence, and allowing the target binding regions of the probe to hybridise to the target nucleic acid molecule;
(ii) a second padlock probe which comprises target-binding regions which are specific for the target nucleic acid sequence.

In an embodiment, the 3′ target binding region of the padlock probe is at least 6 bases shorter than the 5′ target binding region.

In an embodiment, the kit is for detecting a variant target nucleic acid sequence in a target nucleic acid molecule in a sample, and may comprise in part (ii) two or more second padlock probes each comprising target binding regions specific for different variants of the target nucleic acid sequence. In an embodiment, the second padlock probes each comprise a detection sequence by means of which they may be distinguished from one another (that is a detection sequence which is different from that of another second padlock probe).

The kit may comprise one or more further components selected from:
(i) dNTPs, particularly when the dNTPs are provided for use at a concentration in the reaction medium for the extension step of nor more than 10 μM;
(ii) a polymerase, particularly wherein the polymerase is provided for use at a concentration in the reaction medium for the extension step of no more than 0.025 U/uL; and
(iii) a ligase.

### DETAILED DESCRIPTION

The present method provides a high-fidelity and highly-sensitive method for detecting a specific nucleotide sequence (the terms “nucleotide sequence” and “nucleic acid sequence” are used interchangeably herein). The method is particularly useful to detect variants of a target sequence, which may be present in a sample, for example mutant sequences. Particularly, rare target sequences or sequence variants, or sequences or variants present in low abundance, or at low levels, may be detected.

A padlock probe is used to capture a target nucleic acid sequence by gap-fill extension of the target-hybridised 3′ end of the probe to create a complementary copy of the target sequence. Such a padlock probe may be referred to as a “gap-fill padlock” probe. Following the gap-fill extension, the padlock probe is circularised by ligation, and subjected to RCA to create a RCA product comprising multiple repeated tandem complementary copies of the probe, which comprise multiple copies of the target sequence. These amplified target sequences are then probed with a second, target-sequence specific padlock probe, which, upon hybridisation to the target sequence, is in turn circularised and amplified by RCA, generating a second RCA product, by means of which the target sequence may be detected. The generation of a RCA product provides a signal that can readily be detected, and counted. A digital counting readout can be implemented. This allows for digital detection of one reaction product for each detected target molecule. Further, the products of the second RCA reaction are large products which contain multiple (hundreds, and possibly up to the order of a thousand or so) copies of the target sequence, and are of significant size. Such prominent reaction products may readily be collected, with minimal risk for mix-up with any other material in the reaction.

The present method affords a high level of amplification of the target sequence, by means of which the sensitivity of the detection method is improved, rendering it capable of detecting and identifying very rare target sequences. Thus, rare sequence variants, or sequences or variants present at low levels (“low abundance”), may be detected, or identified or discriminated. Further, the method permits accurate quantification of target nucleic acid sequences. For example, the ratios of different target nucleic acid sequences may be determined with high precision, e.g. in the context of determining copy numbers of chromosomes, where target sequences from 2 different chromosomes may be detected and compared. This may be of particular utility in detection of triosomy, for example of chromosome 21, for example in the context of non-invasive prenatal testing (NIPT). The method is shown schematically in FIG. 1.

The use of padlock probes and gap-fill extension to detect target nucleic acid sequences is, as noted above, known in the art. To improve specificity, the present method combines a gap fill extension protocol together with the use of a second padlock probe which specifically targets the gap-infill sequence (which is identical to, or corresponds to the target sequence). In an embodiment, the second padlock probe may also require gap-fill extension before it can be circularised, but this is not a necessary feature.

This second gap fill padlock reaction which targets the filled in sequence in the first RCA product has the effect of further increasing detection specificity as any spurious reaction products from the first gap fill padlocks would not be recognised by the second gap fill padlock probes. This is analogous to the use of nested PCR to increase detection specificity.

Further, in the present method, modifications have been made to the padlock probe and the gap-fill extension and ligation reactions to improve the efficiency with which the padlock probe is extended and circularised, and thereby the efficiency with which the first RCA template for the first RCA reaction is generated. This increases the amount of first RCA product which is generated, and ultimately the amount of signal that can be detected. The efficiency of the method, and thereby its sensitivity is improved.

In one embodiment of the present gap-fill padlock probe-based detection method, the binding of the padlock probe to the target nucleic acid molecule, and the subsequent extension of the hybridised 3′ end of the padlock probe, are performed in two separate steps, to ensure that the padlock probe is fully and properly hybridised at both of its target-binding regions, before the extension reaction takes place. Conveniently, there may be a washing step in between. This is important to regulate the extension step. In this regard, to generate a successful gap-fill product, the extension reaction should stop at the ligation site, and that is achieved by the stable binding of the 5′ end of the padlock probe downstream of the 3′ end of the probe. However, if polymerase and reagents to allow an extension reaction are present at the time of padlock probe binding, the extension from the 3′ end of the gap-fill-ligation padlock probe can occur before the 5′ end is in place, resulting in an over-extended product which cannot be ligated into a DNA circle, reducing the efficiency of detection by such a gap-fill-ligation padlock probe. This is shown in Example 1 below, and FIG. 2.

By separating the gap-fill-ligation probing and extension (polymerization) step, such by-products can be eliminated to a great extent resulting in a better gap-fill efficiency. We can regularly detect around 30-40% of the total targets in complex human genomic DNA samples using such a method.

Whilst the probing step and polymerization step can be combined into one, this reduces the gap-fill-ligation efficiency, and makes the method less suitable for clinical or diagnostic use. It is needed, therefore, to improve the efficiency of a combined probe-binding and gap-fill-ligation protocol to allow for such a use.

The efficiency of target detection via the gap-fill-ligation mechanism may be improved by cycling the gap-fill-ligation step. To allow this to happen, a single-step procedure is needed, wherein the padlock probe is added at the same time as the reagents for the extension (polymerase and dNTPs) and ligation steps (ligase). This is referred to herein as a 1-step gap-fill ligation method. A key factor in such a procedure is to ensure that the 5′ end of the padlock probe has hybridized to its target before the 3′ end extension has proceeded too far. We have identified conditions under which efficient gap-fill extension and ligation occurs. This was achieved by:
a) designing the first padlock probe so that it has a 3′ target binding site that is shorter than the 5′ target binding site by at least be 6 nucleotides.
b) using an extremely low concentration of dNTPs, reducing concentrations from a commonplace 200 μM to no more than 10 μM. As described further below, in some embodiments, the dNTP concentration may be as low as 0.2 μM, that is 1000-fold lower than conventional conditions.
c) maintaining a low concentration of the gap-fill polymerases. As noted above, the polymerase is contacted with the target nucleic acid molecule at a concentration of no more than 0.025 U/μl. Thus, a ten-fold lower concentration of polymerase may be used, compared to standard reaction conditions.

These measures may slow down or delay the extension reaction to ensure that the 5′ end of the probe is stably, or sufficiently, hybridized to the target molecule, so as to minimize or avoid any undesirable over-extension.

Using the above conditions, in another embodiment, a one-step gap-fill-ligation protocol has been elaborated which allows a more efficient generation of ligated gap-filled padlocks. Whilst improvements are gained by increasing the efficiency of the gap fill extension step, further improvements may be gained by cycling the gap-fill extension and ligation steps. Thus, in a further advantageous embodiment, steps (i) and (ii) of the one-step method are cyclically repeated.

It will be understood that such cycling will typically involve thermal cycling. This may be done by procedures well known in the art. Thus, the method may comprise a heating step to denature target nucleic acid molecules, the temperature may then be reduced to allow the first padlock probe to bind (hybridise) to its complementary binding sites in the target molecule (a so-called “annealing” step), following which the hybridized probe is subjected to the extension and ligation steps. This may involve a further temperature reduction and/or increase to optimize the conditions for the subsequent extension and ligation steps, depending on the polymerase and ligase enzyme used. The reaction is then heated again to remove the ligated probe, and start the cycle again, that is to allow further padlock probes to bind, and be extended and ligated, etc. The number of cycles may be varied according to choice, and may depend on the nature of the target nucleic acid, sample, etc. In an embodiment, at least 2 cycles are performed, e.g. at least 3, or 4, for example, from any one of 2, 3, 4, or 5 to any one of 6, 8, 10, 12, 15, 17, 20, 25 or 30 cycles. The number of cycles is not critical and, if desired or appropriate, more can be performed. It has been observed that products accumulate in proportion to the repeated cycle numbers. This leads to an increased generation of first RCA template, and thereby first RCA product, and this increased amplification leads to an increase in the efficiency and sensitivity of the detection method. Whilst such cycling may be advantageous, it is not a requirement of the present method, which includes single-cycle reactions.

At its most general, the method is for detecting a target nucleic acid sequence in a target nucleic acid molecule. The term “detecting” is used broadly herein to include any means of determining the presence of the target nucleic acid sequence in the target nucleic acid molecule. In the present method the target nucleic acid is detected by detecting the presence or amount of the second RCA product which is generated, and can include detecting simply if it is present or not, or any form of measurement of the RCA product. Thus, the RCA product of step (vi) may be detected as the “signal” for the target nucleic acid sequence. Accordingly, detecting the second RCA product in step (vii) includes determining, measuring, assessing or assaying the presence or absence or amount or location of the second RCA product in any way. The presence of a second RCA product (i.e. the confirmation of its presence or amount) is indicative or identificatory of the presence of the target nucleic acid sequence, as the successful generation of the RCA product is ultimately dependent on the presence of the target nucleic acid molecule, and more particularly of the target nucleic acid sequence therein.

Quantitative and qualitative determinations, measurements or assessments are included, including semi-quantitative. Such determinations, measurements or assessments may be relative, for example when two or more different target nucleic acid sequences, or target molecules, in a sample are being detected, or absolute. Accordingly, in an embodiment the method may be for quantifiying or determining the amount of target nucleic acid sequence which is present. The term “quantifying” when used in the context of quantifying a target nucleic acid sequence(s) in a sample can refer to absolute or to relative quantification. Absolute quantification may be accomplished by inclusion of known concentration(s) of one or more control nucleic acid molecules and/or referencing the detected level of the target nucleic acid sequence with known control nucleic acid molecules or sequences (e.g. through generation of a standard curve). Alternatively, relative quantification can be accomplished by comparison of detected levels or amounts between two or more different target nucleic acid molecules, or different target sequences, to provide a relative quantification of each of the two or more different nucleic acid molecules or sequences, i.e., relative to each other. Thus, as noted above, ratios of target nucleic acid sequences present in sample may be determined. Thus, copy numbers of target nucleic acid molecules, e.g. chromosomes, may be compared.

The target nucleic acid sequence is a sequence in any nucleic acid molecule that it is desired to detect, or in other words the target of the assay. As will be described in more detail below, the method may be performed in multiplex to detect two or more different target sequences, for example, in one or more target nucleic acid molecules, and/or a target sequence in two or more target molecules. Thus, to detect target sequences in two or more different target molecules, a multiplicity of first padlock probes may be used, each specific for a different target sequence, that is having target-binding regions which are complementary to binding sites in the target molecule which flank the different target sequences. It will be understood in this respect that the flanking binding sites in the different target molecules will be different for different target sequences, to allow for specific binding of the first padlock probes. Alternatively or additionally, different and separate target sequences in the same target molecule may be detected, for example, different sequences in different genes on a chromosome, again using a multiplicity of different first padlock probes, each specific for a different target sequence. However, as noted above, in a particular embodiment, the method is useful for detecting which of a number of possible different variant sequences is present in a given target molecule, for example whether a wild-type or mutant sequence is present, or which of a number of possible mutants, or different allelic variants, or polymorphisms etc. In such a protocol, a common first padlock probe is used to generate a complementary copy of the target sequence in the ligated first padlock, and it is then determined which variant is present by using multiple different second padlock probes, each specific for a different variant of the target sequence. The step (iv) of contacting the first RCP with multiple second padlock probes, may conveniently be performed in multiplex, in the same reaction medium.

The term “multiple” as used herein means two or more, for example, 3, 4, 5, 6, 8, 9, 10, 15, 20, 25, 30, 40, 50, 60, 70, 80, 90, or 100 or more. Indeed thousands or tens of thousands of padlock probes may be used. The number of padlocks that can be used is not restricted, and can be varied. This will depend on the purpose of the method, the nature of the sample, and the target nucleic acid sequences to be detected, number of possible variants etc. Thus to detect wild-type and mutant variants for examples, the number of different second padlocks will depend on the number of different mutants possible, and may be for example, 2-6, 2-5, 2-4 or 2-3 different second padlocks. It will be appreciated that different aspects may be combined, in order to increase the overall multiplex of the assay. For example in a given sample, the method may be used to detect different variants of different target sequences.

The target nucleic acid sequence may be any sequence it is desired to detect or identify. It may be DNA or RNA, or a modified variant thereof. Thus, the nucleic acid may be made up of ribonucleotides and/or deoxyribonucleotides as well as synthetic nucleotides that are capable of participating in Watson-Crick type or analogous base pair interactions. Thus the nucleic acid may be or may comprise, e.g. bi-sulphite converted DNA, LNA, PNA or any other derivative containing a non-nucleotide backbone.

Typically, the target sequence will be an analyte it is desired to detect, for example a nucleic acid present in a sample, e.g. in a cell or tissue sample or any biological sample etc. Thus, it may be a naturally occurring sequence, or a derivative or copy or amplicon thereof. However, this is not necessary, and the target sequence may instead be a reporter for an analyte of an assay. Reporter nucleic acids may be used or generated in the course of an assay for any analyte, for example a protein or other biological molecule, or small molecule, in a sample. Thus, a reporter nucleic acid may be provided as a tag, or label, for a binding probe for an analyte, and may be detected in order to detect the analyte, for example in an immunoassay, e.g. as in an immunoPCR or immunoRCA reaction. A reporter nucleic acid may be generated in the course of an assay, for example by a ligation reaction in a proximity extension assay, or an extension reaction in a proximity extension assay, or by a cleavage reaction, or such like. Such a reporter target nucleic acid may therefore be a synthetic or artificial sequence.

In an embodiment, the target nucleic acid is a DNA molecule, natural or synthetic. The target nucleic acid molecule may be coding or non-coding DNA, for example genomic DNA or a sub-fraction thereof, or may be derived from genomic DNA, e.g. a copy or amplicon thereof, or it may be cDNA or a sub-fraction thereof, or an amplicon or copy thereof etc.

In another embodiment, the target nucleic acid molecule is a target RNA molecule. It may be an RNA molecule in a pool of RNA or other nucleic acid molecules for example genomic nucleic acids, whether human or from any source, from a transcriptome, or any other nucleic acid (e.g. organelle nucleic acids, i.e. mitochondrial or plastid nucleic acids), whether naturally occurring or synthetic. The target RNA molecule may thus be or may be derived from coding (i.e. pre-mRNA or mRNA) or non-coding RNA sequences (such as tRNA, rRNA, snoRNA, miRNA, siRNA, snRNA, exRNA, piRNA and long ncRNA). one preferred embodiment, the target nucleic acid molecule is a micro RNA (miRNA). In one embodiment, the target RNA molecule is 16S RNA, for example wherein the 16S RNA is from and identificatory of a microorganism (e.g. a pathogenic microorganism) in a sample. Alternatively, the target RNA molecule may be genomic RNA, e.g. ssRNA or dsRNA of a virus having RNA as its genetic material. Notable such viruses include Ebola, HIV, SARS, SARS-CoV2, influenza, hepatitis C, West Nile fever, polio and measles. Accordingly, the target RNA molecule may be positive sense RNA, negative sense RNA, or double-stranded RNA from a viral genome, or positive-sense RNA from a retroviral RNA genome.

Where the target molecule is an RNA molecule, the method may comprise a preliminary step of generating a cDNA copy of the target RNA molecule. The cDNA molecule is then contacted with the first padlock probe in step (i).

Alternatively, a target RNA molecule may directly be contacted with the first padlock probe. In other words, the first padlock probe may bind directly to the target RNA molecule. For such a method, the polymerase used for the gap-fill extension step would have reverse transcriptase activity, particularly a polymerase capable of reverse transcriptase activity that does not have strand displacing activity.

For the detection of a variant target sequence, a first padlock probe is used which is capable of capturing any possible variant of a given target sequence. The first padlock probe is thus not selective, or specific, for any particular variant. However, to ensure capture of a desired target sequence it will be designed to specifically bind to the target molecule at sites which allow all variants of the target sequence to be captured, that is at sites which flank the target sequence, and which are common between different variants. By way of example, the variant sequence may be a mutation or a polymorphism at a particular position or locus in a gene. The target sequence may thus be a sequence which includes or comprises that variant position or locus, and different target sequences may be distinguished by having different bases at that position or locus. The first padlock probe may be designed to have binding sites which are complementary to flanking sequences in the target molecule which are shared or conserved (i.e. common) between the different variants.

Accordingly, in an embodiment, a first padlock probe may be viewed as a common probe, or as generic to a group of target sequences, or a group of variant target sequences. The first padlock probe may thus have target-binding regions which are complementary to binding sites (i.e. flanking regions) in the target molecule which are common to different target sequences (that is common binding sites which flank different target sequences, or different variants of a target sequence). Alternatively put, the first padlock probe may have target binding regions which are common, or generic, for different target sequences, or different variants, or common for a group of target sequences or group of variants. That is the first padlock probe may have target binding regions which are capable of hybridising to complementary binding sites in the target molecule which are common to different target sequences, or to different variants of target sequences. The second padlock probe is, however, specific to the target sequence, or to different variants of a target sequence, as described further below.

In a different embodiment, the first padlock probe may be specific for a particular target nucleic acid sequence. Thus, for example different first padlock probes may be used, each specific for a different target sequence. This may have utility for diagnostic testing, e.g. NIPT, where different sequences may be detected, for example to detect different chromosomes (and determine their copy number, for example to detect a triosomy) or in any situation where it is desired to detect one or more specific target sequences.

A padlock probe may alternatively be defined as a circularisable probe. The use of padlock or circularisable probes is well known in the art, including in the context of RCA reactions. A circularisable probe comprises one or more linear oligonucleotides which may be ligated together to form a circle. Padlock probes are well known and widely used and are well-reported and described in the literature. Thus, the principles of padlock probing are well understood and the design and use of padlock probes is known and described in the art. A padlock probe is typically a linear circularisable oligonucleotide which hybridizes to its target nucleic acid sequence or molecule in a manner which brings 5′ and 3′ ligatable ends of the probe into juxtaposition for ligation together, either directly, or as described above, indirectly, with a gap in between. It is this latter, indirect, or “gap-fill” configuration which is used for the first padlock probe in the present method. The second padlock probe may have ligatable ends which are ligated directly or indirectly. By ligating the hybridized 5′ and 3′ ends of the probe, the probe is circularized. It is understood that for circularization (ligation) to occur, the ligatable 5′ end of the padlock probe has a free 5′ phosphate group.

To allow the juxtaposition of the ends of the padlock probe for ligation, the padlock probe is designed to have the target-binding sites at or near its 5′ and 3′ ends. That is, the regions of complementarity which allow binding of the padlock probe to its target lie at or near the ends of the padlock probe. In the case of the first padlock probe, these regions of complementarity allow specific binding of the padlock probe to its target molecule by virtue of hybridization to specific sequences, binding sites, in the target molecule, which flank the target sequence.

To allow ligation, the 3′ and 5′ ends which are to be ligated (the “ligatable” 3′ and 5′ ends) are hybridized to the target molecule or sequence, which acts as the ligation template. The ligatable ends of a padlock probe may be brought into juxtaposition for ligation in various ways, depending on the probe design. Where the target-binding sites are located at the ends of the padlock probe, the binding of the padlock probe may bring the ends into said juxtaposition. Where the complementary binding sites in the target molecule or sequence lie directly adjacent (or contiguous) to one another, the ends of the padlock probe will hybridise directly adjacent to each other (i.e. with no gap) and may be ligated to each other directly. Thus, in this case the ligatable ends of the probe are provided by the actual ends of the probe. Such a configuration may be adopted for the second padlock probe (although the method is not limited to this). However, the first padlock probe is a gap-fill padlock probe, and hence the binding sites at the ends of the padlock probe do not hybridise to adjacent binding sites, but rather to non-adjacent (non-contiguous) binding sites in the target molecule which flank the target sequence. In such an arrangement, the 5′ ligatable end of the probe is provided by the actual 5′ end of the probe. However, the ligatable 3′ end of the probe is generated by extension of the hybridized 3′ end of the probe, using the target sequence as extension template to fill the gap between the hybridized ends of the probe. The extension reaction brings the extended 3′ end of the probe into juxtaposition for ligation. In this case, the ligatable 3′ end of the probe is thus the extended 3′ end of the probe.

In other embodiments the ligatable 3′ and/or 5′ ends may be created, or generated, by cleavage. Thus, where the target binding sites do not lie at the ends of the padlock probe, but rather are located internally of the ends, near (rather than at) the ends of the probe, the probe will hybridise to the target in a manner in which there are unhybridised nucleotides located at the probe ends. In other words, after probe hybridization there is an overhang, or flap, or unhybridised additional sequence at one or both ends of the probe. This will prevent ligation of the hybridized probe, or indeed extension, if the unhybridised sequence is at the 3′ end. These unhybridised regions or nucleotides may be removed by cleavage, particularly by enzymatic cleavage, when the probe is hybridized to its target (i.e. by cleavage of the hybridized probe).

Hybridisation of padlock probes with an internal 5′ target binding site will result in a structure with a so-called 5′ flap. Padlock probes designed in this manner are known in the art, as are procedures and enzymes for cleaving them. Any enzyme capable of performing a reaction which removes a 5′ flap may be used in this step, i.e. any enzyme capable of cleaving, degrading or digesting a 5′ single-stranded sequence which is not hybridised to a target nucleic acid molecule, but typically this will be an enzyme with 5′ nuclease and/or structure-specific cleavage activity.

A structure-specific cleavage enzyme is an enzyme capable of recognising the junction between single-stranded 5′ overhang and a DNA duplex, and cleaving the single-stranded overhang. Such enzymes are known in the art and include flap endonucleases (FENS), which are a class of enzymes having endonucleolytic activity and being capable of catalysing the hydrolytic cleavage of the phosphodiester bond at the junction of single- and double-stranded DNA. For example, the enzyme may be a native or recombinant archaeal FEN1 enzyme from P. furiosus (Pfu), A. fulgidus (Afu), M. jannaschii (Mja) or M. thermoautotrophicum (Mth).

Enzymes having 5′ nuclease activity include enzymes with 5′ exonuclease and/or 5′ endonuclease activity, and again such enzymes are known in the art, e.g.

Taq DNA polymerase and the 5′ nuclease domain thereof, or Exonuclease VIII. Other examples are RecJf and T5 exonuclease.

For cleavage of an unhybridised 3′ end (or 3′ flap) an enzyme with 3′ nuclease activity may be used. This may be 3′ exonuclease or 3′ endonuclease activity. This may be provided by a polymerase with 3′ exonuclease activity, or the 3′ exonuclease domain thereof, or by a separate exonuclease enzyme, e.g. exonuclease I, or by an endonuclease. By way of representative example, the enzyme may beT4 DNA polymerase, T7 DNA polymerase, DNA polymerase I, Klenow fragment of DNA polymerase I, Pyrococcus furiosus (Pfu) DNA polymerase and/or Pyrococcus woesei (Pwo) DNA polymerase.

In particular, a polymerase with 3′ exonuclease activity may be used for the step of extending the hybridised 3′ end of the probe in step (ii)—such an enzyme will remove the unhybridised 3′ nucleotides to leave a hybridised 3′ end before the extension reaction takes place. A polymerase with 3′ exonuclease but without strand-displacing activity is desirable. In the case of a cycling protocol, a thermophilic polymerase should be used. These include: Q5/Q5U DNA polymerase, Phusion/Phusion U DNA polymerase, Taq DNA polymerase, Stoffel DNA polymerase, Pwo DNA polymerase, Kappa DNA polymerase, and SuperFi DNA polymerase. For protocols without cycling, including the 1-step method without cycling, T4 DNA polymerase, T7 DNA polymerase or DNA polymerase I may be used.

The use of a 3′ flap may be advantageous in the context of a 1-step gap fill protocol, in that by requiring unhybridised nucleotides at the 3′ end of the probe to be removed before extension can take place, a delay may be created to the extension reaction. This may allow the 5′ end of the probe to become hybridised before the extension reaction takes place, or before the extension reaches the hybridised 5′ end (or hybridised 5′ target binding site of the probe).

The positioning of the target binding site away from the 3′ or 5′ end of the padlock probe will determine the length of the 3′ of 5′ flap. Generally speaking, it is preferred for the 3′ target binding site to be reasonably close to the 3′ end, for example within 7 or 6, or fewer nucleotides of the 3 end, e.g. 5, 4, 3, 2 or 1 nucleotide of the end. For the 5′ target binding site, a longer distance may be tolerated. In an embodiment, “near” to a 5′ or 3′ end of the probe means within 12 nucleotides or less of the end, e.g. within 10, 9, 8, 7 or 6 nucleotides of the end. In the case of the 3′ end, this may for example be within 8, 7, 6, 5, 4, 3, 2 or 1 nucleotides of the end or less, e.g. within 6 nucleotides or less.

In another embodiment, the 3′ end of the first padlock probe may comprise a hairpin structure which comprises the 3′ target binding region. In other words, the 3′ target binding region may be at least partially comprised within a hairpin structure at the 3′ end of the padlock probe. When the probe hybridises to the target molecule, strand displacement will cause the hairpin to open, and for the 3′ end of the probe to hybridise to the target molecule, such that it can be extended. This provides an alternative way of providing an additional extension delay (beyond that afforded by the measures indicated in step (i) above), to ensure that the 5′ end of the probe is hybridised before the extension takes place, or reaches the hybridised 5′ end (or more particularly the hybridised 5′ target binding site).

Padlock probes may be provided in 2 or more parts that are ligated together. This may involve the provision of an additional ligation template, for example in the case of a 2-part probe, where each part comprises only one target-binding region, and the other end of each part hybridizes to a common ligation template. In another embodiment, a 2-part padlock may take the form of a “connector” oligonucleotide with two target-binding regions at or near the 5′ and 3′ ends respectively, which hybridise to the target with a gap in between them, and a gap oligonucleotide which hybridizes in the gap between the ends. The gap oligonucleotide may partially or fully fill the gap. In the case of a gap-fill padlock probe, the 3′ end which is extended may be the hybridized 3′ end of the gap oligonucleotide or the backbone oligonucleotide. In a typical embodiment, however, the padlock is provided as a single circularisable oligonucleotide, whether as a gap fill padlock (which is required for the first padlock probe), or not.

In particular embodiments the padlock probe does not have secondary structure, and more particularly does not comprise intramolecular double-stranded regions or stem-loop structures. However, dumbbell probes, which do have secondary structure, are a particular sub-type of padlock probe. The dumbbell probe comprises two stem-loop structures, joined stem to stem, wherein one of the “loops” is not closed, but is open with free 5′ and 3′ ends available for ligation to each other. This “open loop” functions as the target-binding domain of the probe. The closed loop functions simply as a spacer to join the end of the duplex (stem). In other words it can be seen as a padlock probe with a region of duplex formed between complementary sequences (regions) of the padlock. The region of duplex functions as a signalling domain to which an intercalating agent can bind. Thus the “open loop” of a dumbbell probe may comprise the target-binding regions of complementarity.

A variant sequence to be detected in the method may comprise one or more variant bases. Thus, it may be a single nucleotide variant, e.g. a single nucleotide polymorphism (SNP) or mutation, or it may comprise two or more bases. Thus, a variant sequence may comprise a stretch of nucleotides, 2 or more bases of which may be variant. The variant bases may be contiguous or non-contiguous.

The length of the target sequence is not critical, and may vary according to circumstance, and the nature of the target molecule, the target sequence, or the variant position or locus. A target sequence may thus be, by way of representative example only, 1 to 10, e.g., 1-15, 1-12. 1-10, 1-8, 1-7 or 1-6 nucleotides long. In certain embodiments however, a target sequence longer than a single nucleotide may be beneficial, to improve specificity, and in such embodiment, the target sequence may be from any one of 2, 3, 4, 5, or 6 to any one of 6, 7, 8, 9, 10, 12, 15 or 20 nucleotides long. Exemplary target sequences may thus be 4-10, 4-8, 4-7, 4-6, 5-8, 5-7, or 6-8 nucleotides long for example.

It will be understood that the length of the target sequence will correspond to the length of the gap between the ends of the hybridised target binding sites of the first padlock, and thus that the length of the gap may be any of the ranges set out above.

In the one-step gap-fill-ligation method set out above, the features of step (i) act to delay or slow down the extension reaction, to avoid over-extension which would prevent or reduce the ligation of the probe.

The first such feature is to design the first padlock probe with a 3′ target-binding site which is at least 6 nucleotides shorter than the 5′ target binding site. In an embodiment, the 3′ target binding site is at least 7 or 8 nucleotides shorter than the 5′ binding site. By way of representative example the 3′ target binding site be at least 6 nucleotides long, e.g. at least 7, or 8 nucleotides long, e.g. 6-20. 6-18, 6-15, or 6-12 nucleotides long. The 5′ target binding site may be at least 12 nucleotides long, e.g. at least 13, 14, or 15 nucleotides long, for example 12-30, 12-25, 12-20, 12-18, or 12-15 nucleotides long. This differing length helps to ensure that the 5′ target binding site is stably hybridised before or at least at the same time as the 3′ target binding site.

Secondly, the reagents for the extension reaction, namely the dNTPs and polymerase enzyme are provided in, or added to, the reaction mixture for the combined probe-binding, extension and ligation steps at low levels, much lower than used in conventional reactions in the art. As noted above, for dNTPs, this can be up to 1000-fold lower. The concentration of dNTPS used may depend on the polymerase which is used.

The polymerase enzyme for the extension step is provided at a concentration of no more than 0.025 U/μl. More particularly, the concentration is no more than 0.02, 0.015, 0.01, 0.005, 0.004, 0.003 or 0.0025 U/μl. The concentration may be e.g. 10-fold lower than conventionally or normally used. Whilst this may slow down and reduce the efficiency of the extension reaction in itself, this may nonetheless result in an acceptable level of extension, and in the context of the method as a whole, an efficient gap-fill-ligation step, which generates the first RCA template for the first RCA reaction in the detection protocol. For example 0.0025 U/μl polymerase may be used.

The polymerase used in the extension reaction is a polymerase enzyme or domain or part thereof without strand-displacing activity. Non-strand displacing prolymerases are known in the art, and include for example Q5/Q5U DNA polymerase, Phusion/Phusion U DNA polymerase (Thermo Fisher), T4 polymerase, T7 polymerase, Pwo DNA polymerase, Kappa DNA polymerase, SuperFi DNA polymerase, and Pfu DNA polymerase. Taq polymerase is not strand-displacing, but has a 5′ exonuclease activity which may not be desirable. Taq derivatives and fragments without this 5′ exonuclease activity are available and may be used, including for example the Stoffel fragment. In an embodiment a high fidelity DNA polymerase is used. This includes Stoffel and Phusion DNA polymerase, which are widely available from various sources.

As noted above, the concentration of dNTPs to be used may depend on the polymerase which is used. For example, for the Stoffel fragment, the concentration may be no more than 1 μM, in particular no more than 0.5 μM, and more particularly no more than 0.3, or 0.25 μM.

Where the polymerase is Phusion polymerase the dNTPs may be provided at a concentration of no more than 10 μM, particularly nor more than 3 μM, and more particularly no more than 1 μM.

To perform the one-step method the target nucleic acid is contacted with the first padlock probe and other reagents. The sample containing the target nucleic acid molecule, or a part or fraction or aliquot thereof may be contacted with the reagents. For example, for in situ analyses, a cell or tissue sample may be contacted with the reagents directly. The sample may be prior-treated or processed prior to the contact, e.g. by fixation. In other embodiments the target nucleic acid may first be separated, or removed from the sample. Procedures for extracting or purifying nucleic acids, e.g. DNA, from various types of sample are well known in the art. For example, the nucleic acids may be isolated from cells, or from cell-free samples, such as plasma. It may in some cases also be desirable to fragment nucleic acid molecules. Procedures for this are known in the art, and include specific digestion, e.g. using nucleases, including restriction enzymes for example, or by non-specific means, such as shearing.

The contacting step prepares a reaction mixture for the probe-binding, extension and ligation reactions.

To perform the probe-binding, extension, and ligation reactions, the first padlock probe is incubated with target nucleic acid molecule, dNTPs, polymerase and ligase. To allow for probe-binding there may be an initial heating step, for example to denature a double-stranded nucleic acid molecule.

The reaction mixture may be incubated in conditions appropriate to facilitate or enable padlock probe binding (the so-called “annealing” step). If there has been a preceding denaturation step this may involve a reduction in temperature. Conditions for these steps are known in the art, and are within the routine skill of the skilled practitioner in the art to select or design. The annealing temperature may be selected to facilitate or optimise the annealing of the 5′ target binding region of the probe. For example, an annealing temperature of 50-65° C. may be used, e.g. 53 to 60° C., or more particularly 55 to 60° C.

The annealing temperature may be reduced for the extension step. Again the appropriate conditions can be selected according to what is known in the art, and the particular reagents, e.g. enzymes used. For example, after the initial annealing step, the temperature may be reduced to 28-40° C., e.g. 28-35, 30-35, 28-33, 30-33, 28-33, or 30-32° C. etc. Lower temperatures, e.g. around 32° C., may be favourable to prevent or reduce over-extension.

The extension temperature may be selected to allow or facilitate hybridisation of the 3′ target binding site of the probe, followed by extension. Thus, by selecting appropriate conditions, including temperature, a balance may be obtained, which allow stable hybridisation of the 5′ target binding site of the first padlock probe, followed by hybridisation of the 3′ target binding sites and extension the hybridised 3′ end.

Once the extension reaction has taken place, and optionally any cleavage step that may be required to remove a 5′ flap, the padlock probe is ligated to circularise it, and thereby generate the template for the first RCA reaction. The ligation is template by the target nucleic acid molecule. Any convenient ligase may be employed, and representative ligases of interest include, but are not limited to, temperature sensitive ligases such as bacteriophage T4 DNA ligase, bacteriophage T7 ligase, and E. coli ligase, and thermostable ligases such as Taq ligase, Tth ligase, Ampligase®, Pfu ligase and 9°N™ DNA Ligase.

Suitable conditions for ligation are known in the art, and any reagents that are necessary and/or desirable may be combined with the reaction mixture and maintained under conditions sufficient for ligation. It will be evident that the ligation conditions may depend on the ligase enzyme used in the methods of the invention. Thus, for example, Ampligase may be used, and the temperature may be increased following the extension step, for the ligation step.

Conveniently, the method may be performed in a thermal cycling instrument. This permits a ready control of the temperature changes. Thermal cyclers permit ramping speeds, that is the speed at which the temperature is changed, to be controlled, and it has been found that ramping speeds may also be selected to optimise the reactions, and improve the yield of the gap-filled ligated first padlock probes. For example 100% of the ramping speed of the instrument may be used (e.g. 3.3° C./s). Slower ramping speeds, e.g. 4% ramping speed (e.g. 0.13° C./s), may improve yield, but may require longer incubation times.

The conditions for the gap-fill extension and ligation reactions may be optimised by routine experimentation according to principles known in the art. Thus, temperature, buffers, time of incubation, ramping speed etc. may be adjusted to find the optimal conditions.

It has been observed that the inclusion of a crowding agent in the reaction mixture may be beneficial. Suitable crowding agents are known in the art, and include for example PEG, glycerol or a gel such as Sephadex.

For the 2-step method, the probe-binding step is performed in the absence of polymerase, but otherwise the conditions and details may be as set out above. For the 2-step reaction, the probe-binding step is performed in the absence of polymerase, but otherwise the conditions and details for the contacting of the target nucleic acid with the padlock probe, and incubation etc., and the extension and ligation reactions may be as set out above. The other reagents for the extension reaction (e.g. dNTPs) and the ligation reaction (e.g. ligase) may be contacted with the hybridised probe/target nucleic acid after the probe has been bound, together with the polymerase, optionally after a washing step to remove unbound padlock probes. Alternatively the reagents other than the polymerase may be contacted with the target nucleic acid molecule together with the first padlock probe, and the polymerase may be added subsequently, to start the extension reaction after the padlock probe has hybridised. The ligase for the ligation step may be added after extension, or may be included with the polymerase and/or other extension reagents. Thus, other reagents (that is reagents other than the polymerase) may be added before, during, or after the addition of polymerase.

Once the gap-filled ligated first padlock probe has been generated, it is subjected to the first RCA reaction. Before the RCA reaction there may be an optional washing step, for example if the method is performed in a heterogenous or solid phase format, e.g. when the sample is fixed or immobilised on a solid support. This may serve to remove unligated or unhybridised probes.

Alternatively or additionally, there may be clean-up step before the first RCA reaction using an exonuclease to degrade, and thereby remove unbound and/or unligated, and hence non-circularised, probes, and where appropriate excess or unwanted nucleic acid, for example excess genomic DNA. Such a step may be particularly be performed in homogenous, or solution phase, formats. Exonucleases for performing such a clean-up are known in the art, for example, exonuclease I, Ill or lambda, or mixtures thereof. Further, exonuclease activity of a polymerase may be used to achieve this, e.g. 3′ exonuclease activity. Such a clean-up by exonucleolysis may conveniently be performed by adding an enzyme having exonuclease activity after the ligation step. This could be, or could include, the polymerase enzyme used for the RCA reaction. Alternatively, an enzyme with exonuclease activity may be added before the ligation step to remove unhybridised probes—in such a case the enzyme should be selected to have strict single-stranded specific activity, for example Exol, RecJf.

As noted above, RCA reactions are well known in the art, and hence the conditions for this step may be designed or selected according to protocols and principles known and described in the literature. A strand-displacing polymerase enzyme such as Phi29 or derivatives thereof is used. The primer for the RCA reaction may be added to the reaction mixture, or may be pre-hybridised to the first padlock probe. The binding site for the RCA primer may be provided in a region of the padlock probe which is different to the target binding regions (i.e. in the backbone region of the padlock). In some cases the target nucleic acid molecule may serve as or provide the primer, for example in in situ applications. If necessary, 3′ exonuclease action of the polymerase, or a separate exonuclease may be used to digest the target nucleic acid to provide a hybridised 3′ end suitable to act as the RCA primer for RCA of the circularised padlock probe.

Reagents for the RCA reaction may be added after a clean-up step, or one more reagents may be included in the clean-up step, and other reagents to initiate the RCA reaction may be added after. Separate exonucleases added for clean-up may be inactivated, for example by heating or treatment with a protease (e.g. proteinase K) prior to commencement of the RCA, and reagents for RCA may be added thereafter.

After the first RCA reaction, there may be a step to inactivate the polymerase enzyme used, e.g. by heating, prior to the subsequent steps (e.g. prior to addition of the second padlock probe, or prior to ligation thereof.

The RCA reaction produces a concatemeric first RCA product (RCP) comprising multiple repeat copies of the complement of the gap-filled and ligated first padlock probe. It therefore comprises multiple repeat copies of the target sequence which was copied into the gap-filled probe. In this way the target sequence is amplified. The target sequence provides a binding site for the second padlock probe. The first RCP thus provides multiple binding sites for the second, target sequence-specific, padlock probe. Thus, more particularly, multiple copies of a given second padlock probe may hybridise to the first RCP. It will however be understood that not every available binding site in the first RCP needs be occupied or bound by a second padlock probe — it suffices that a number or multiplicity of binding sites in the first RCP are bound by the second padlock probe. More particularly, a target sequence in at least one monomer of the concatemeric first RCP is bound by a second padlock probe, but more particularly, in at least 2, 4, 6, 8, 10, 12, 15, 20, 25, 30, 40, 50, 80, 100, 150, or 200 or more monomers.

The method relies upon multiple probes being able to hybridise to the first RCA product. Accordingly, it will be understood that the first RCA product needs to be available for probe hybridisation. This requirement is a feature of all RCA-based detection methods, where an RCA product is detected by hybridising a probe, e.g. a detection probe, to the product, and is well understood in the art. Thus, it may be advantageous for the first RCA product to have low secondary structure. However, this feature may be compensated for by performing the method in conditions which favour hybridisation, according to principles well known in the art. Thus, for example, the step of binding the second padlock probes in the method can be performed in the presence of formamide e.g. in buffers containing formamide, although generally speaking it will be desirable to remove formamide before the second RCA reaction is performed, for example by washing. In formats where washing is not used, e.g. in-solution formats DMSO or betaine may alternatively be used, if desired, to enhance binding of the second padlock probes.

The second padlock is specific for the target sequence and thus is used to identify or detect the target sequence, or a variant thereof. The second padlock probe thus comprises target-specific binding regions which are specific for a particular target sequence or for a particular variant. The target binding regions are thus designed to be complementary to a specific sequence in the target sequence, or to distinguish or discriminate between different target sequences or variants. The design of variant-specific, or allele-specific, probes is known in the art, and thus the binding regions of the second padlock can readily be designed to detect or identify a desired target sequence or variant. The second padlock probe accordingly allows genotyping of the target sequence in the first RCP and may be referred to as a genotyping padlock probe.

As discussed above, in the case of detecting a variant target sequence, two or more second padlock probes may be used, each specific for a different variant sequence and it may be detected which of these produces a second RCP, in order to detect, or identify, which variant is present.

As noted above, there may be one or more variant bases in a variant sequence, and these be contiguous or non-contiguous.

The position of the variant base(s) in the target sequence may affect the efficiency of the preceding gap-filling ligation reactions and/or the sensitivity or accuracy of the detection by the second padlock probe. For example if a variant base is located in a particular position of the target sequence, and hence in a particular position in the gap, this may affect whether erroneous (i.e. non-specific) products are produced in the gap-fill ligation reaction.

In one embodiment the variant target nucleic acid sequence comprises a single variant base, and the variant base is not located at the position corresponding to the first or the last base of the gap between the hybridised ends of the first padlock probe (or in other words, not at the first or last position of the target sequence).

The skilled practitioner would know how to design the first and second padlock probes with respect to optimise the gap-fill-ligation and subsequent detection reactions, and this may involve routine trial and error, for example, with respect to gap size (length of target sequence) and/or position of the variant bases.

The second padlock probe may have any design or configuration discussed above. Thus, it may be ligated directly or indirectly, and the target binding regions may lie at or near the respective 5′ and 3′ ends as discussed above, and they may hybridise to adjacent or non-adjacent binding sites in the target sequence. Thus the second padlock may be a gap filling padlock or not, and may or may not generate 5′ and/or 3′ flaps upon hybridisation to the first RCP. It may be in 1 or more parts.

However, in an embodiment the second padlock probe is single circularisable oligonucleotide comprising 5′ and 3′ target-binding regions at its respective 5′ and 3′ ends. In a further such embodiment, the second padlock probe may hybridise such that the hybridised 5′ and 3′ ends are directly adjacent and may be ligated directly.

In an alternative embodiment, the second padlock probe may hybridise such that the hybridised 5′ and 3′ ends are not directly adjacent and require a gap-fill reaction, e.g. an extension step, before ligation can occur. In such an embodiment, the gap fill design of the second padlock probe is helpful for quality control of such probes, for example by sequencing the second padlock probe to exclude the incorporation of irrelevant sequences. This same feature can also be helpful in routine use by confirming that the intended sequence has been detected via the incorporated gap fill fragment, further guaranteeing target selectivity. The gap-fill reaction for the second padlock probe may be full or partial. In other words, the gap which is filled-in in the case of the second padlock probe may be the full length of the target sequence, or less than the full length, such that only a part of the target sequence is copied into the second padlock probe. Accordingly, the first and second gap-fill steps (e.g. gap-fill extension reactions) may be fully or partially overlapping.

For detection of a variant base, the 5′ or 3′ end (or more generally target binding region) of the second padlock probe may, for example be designed to hybridise, or be complementary, to the variant base, or to a base immediately preceding or following the variant base. As noted above, the skilled person knows how to design the binding regions of the second padlock probe to achieve specific detection of a particular variant base or sequence. Typically, a single variant base will be placed at the 3′ target binding site of the second padlock probe, or more particularly the last base of the 3′ target binding site, but the target base can also be present at bases no more than 6 nt upstream of the 3′ target binding site or at the 5′ end of the target binding site or no more than 6 nt upstream of the end of the 5′ target binding site.

Once the second padlock probes are hybridised, they are ligated to circularise them. This may be performed as discussed above.

After ligation of the second padlocks, the second RCA is performed using the ligated padlock probes as RCA template. The second RCA reaction may be primed by a primer which is separately added to the reaction mix. As discussed above, reagents, procedures and protocols for conducting RCA reactions are known in the art and can be employed here. If desired, there can be one or more intervening washing and/or clean-up steps to remove any unbound and/or unligated second padlock probes, if desired. For example washing may take place after hybridisation of the second padlock probes to the first RCP and/or after ligation of the probes. This may readily be achieved for example by carrying out the method in a solid phase format. In a solution-phase format, enzymes may be used to degrade un-reacted second padlocks, for example exonucleases as described above. In general, a clean-up step may be applied after each probe circularisation reaction to remove remaining unreacted probes and sample nucleic acid sequences, but sparing successfully circularised probes. The exonuclease clean-up step is most suitable after the first padlock ligation reaction. In the event that an exonuclease clean-up step is applied after the second padlock ligation reaction, this must be carried out in such a way that the first-generation RCA product is spared.

The method herein requires a first RCA step, templated by the gap-filled and circularised first padlock probe, and at least a second RCA step, templated by the circularised second padlock probe, which may or may not be a gap-fill padlock probe. As noted above, the method may comprise further RCA steps, to generate a third, or further generation RCA product, using third, or fourth padlock probes, and so on, each targeting the target nucleotide sequence. In other words, if desired, steps (iv) to (vi) or (iv) to (vii) may be repeated. The final generation RCA product may be detected.

It will be understood that such further generations of RCA may act to increase the signal which is ultimately detected. This may accordingly result in increased signal amplification. However, it will be also be understood that where the second and further padlock probes are gap-fill padlocks, this will result in the increased synthesis of copies of the target nucleotide sequence, or of a part thereof (depending on whether successive gap-fill reactions are fully or partially overlapping). In other words, the method may result in a clonal production of large numbers of the filled-in sequence. In still other words, the filled-in sequence (i.e. the target nucleic acid sequence, or a part thereof) may be amplified. This can be useful for preparative reactions. If multiple successive (i.e. two or more) second and further padlock probe ligation steps involve gap-fill of the same or overlapping sequences, then more copies of a specific target sequence can be generated.

The method may be carried out in heterogenous or homogenous formats. That is, it may be performed on a solid phase (or support), or in solution or suspension (i.e. without a solid phase or support), or indeed both, since a solid phase may be introduced at a later stage, for example at the step of detecting the second RCP, or at the stage of generating the second RCP, etc.

The format of the method may be selected based on the nature of the sample, or the target nucleic acid molecule, or the desired readout or detection technology used. For example, for liquid or liquefied or solubilised samples, or for isolated or purified nucleic acids etc., or for the detection of non-nucleic analytes in samples (for example detection of proteins in serum or plasma, or other body fluids etc.) a solution phase format may be adopted. On the other hand, to detect target sequences in solid samples, such as tissues or cells, for example for in situ detection, a solid phase format may be used. It may also be desired to immobilise the sample or target nucleic acid for other reasons, e.g. for a particular assay format, or simply according to choice. Thus, a sample of isolated cells may be fixed on support, or nucleic acids may be isolated or captured from a sample onto a solid support, or such like. In another embodiment, the primer for the second RCA reaction may be immobilised on solid support.

In one embodiment the method may be for the localised detection of target nucleic acid sequences. “Localised” detection means that the signal giving rise to the detection of the nucleic acid is localised to the nucleic acid, in this case the second RCP is localised to the target nucleic acid. The nucleic acid may therefore be detected in or at its location in the sample. In other words the spatial position (or localization) of the nucleic acid within the sample may be determined (or “detected”). This means that the nucleic acid may be localised to, or within, the cell in which it is located or expressed, or to a position within the cell or tissue sample. Thus “localised detection” may include determining, measuring, assessing or assaying the presence or amount and location, or absence, of nucleic acid in any way.

In a particular embodiment, the method may be used for the localised, particularly in situ, detection of a target nucleic acid sequence. More particularly, the method may be used for the localised, particularly in situ, detection of a nucleic acid, particularly mRNA, in a sample of cells.

As used herein, the term “in situ” refers to the detection of a target nucleic acid sequence in its native context, i.e. in the cell or tissue in which it normally occurs. Thus, this may refer to the natural or native localization of a target nucleic acid sequence, e.g. RNA. In other words, the nucleic acid may be detected where, or as, it occurs in its native environment or situation. Thus, the nucleic acid is not moved from its normal location, i.e. it is not isolated or purified in any way, or transferred to another location or medium etc. Typically, this term refers to the nucleic acid as it occurs within a cell or within a cell or tissue sample, e.g. its native localization within the cell or tissue and/or within its normal or native cellular environment. In particular, in situ detection includes detecting the target nucleic acid (e.g. RNA, especially mRNA) within a tissue sample, and particularly a tissue section. In other embodiments the method can be carried out on a sample of isolated cells, such that the cells are themselves are not in situ.

In other embodiments, as noted above, the detection is not localized, or not in situ. In still other embodiments, the method can be carried out in solution. In particular the nucleic acid can be in solution. Thus, for example, the method can be performed on a sample comprising isolated nucleic acid.

The target nucleic acid molecule may be present within a sample. The sample may be any sample which contains any amount of nucleic acid, from any source or of any origin, in which it is desired to detect a target nucleic acid sequence in a target nucleic acid molecule. A sample may thus be any clinical or non-clinical sample, and may be any biological, clinical or environmental sample in which the target nucleic acid molecule may occur.

The sample may be any sample which contains a target nucleic acid molecule, and includes both natural and synthetic samples, that is, materials which occur naturally or preparations which have been made. Naturally occurring samples may be treated or processed before being subjected to the methods herein. All biological and clinical samples are included, e.g. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared or they may be prior-treated in any convenient way e.g. for storage.

Representative samples thus include any material which may contain a target nucleic acid molecule, including for example foods and allied products, clinical and environmental samples. The sample may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue green algae, fungi, bacteria, protozoa etc., or a virus. The cells may be for example human cells, avian cells, reptile cells etc., without limitation.

Representative samples thus include whole blood and blood-derived products such as plasma, serum and buffy coat, blood cells, urine, faeces, cerebrospinal fluid or any other body fluids (e.g. respiratory secretions, saliva, milk, etc.), tissues, biopsies, cell cultures, cell suspensions, conditioned media or other samples of cell culture constituents, etc. The sample may be pre-treated in any convenient or desired way to prepare for use in the method, for example by cell lysis or purification, isolation of the nucleic acid, etc.

In one embodiment the sample comprises microbial cells or viruses which have been isolated from a clinical sample or from a culture of a clinical sample. In such a sample the target nucleic acid molecule may be a nucleotide sequence present in a microbial cell, e.g. a nucleotide sequence which is characteristic for, or discriminatory or identificatory of a microbial cell or virus, at any level, e.g. at type, group, class, genus, species or strain level.

In another embodiment the sample may contain cell-free DNA. The sample may be sample such as plasma or serum which directly contains cell-free DNA, or the cell-free DNA may be isolated.

In another embodiment the sample may contain exosomes.

For localised in situ detection the sample may be any sample of cells in which a nucleic acid molecule may occur, to the extent that such a sample is amenable to localized in situ detection. This may be a sample in which the nucleic acid is present at a fixed, detectable or visualizable position in the sample. The sample will thus be any sample which reflects the normal or native (“in situ”) localization of the nucleic acid, e.g. RNA, i.e. any sample in which it normally or natively occurs. Such a sample will advantageously be or comprise a cell or group of cells such as a tissue. Examples are samples such as cultured or harvested or biopsied cell or tissue samples in which the nucleic acid may be detected to reveal the qualitative nature of the nucleic acid, i.e. that it is present, or the nucleotide sequence of the nucleic acid or the presence and/or identity of one or more nucleotides in the nucleic acid, and localization relative to other features of the cell. The sample of cells may be freshly prepared or may be prior-treated in any convenient way such as by fixation or freezing. Accordingly, fresh, frozen or fixed cells or tissues may be used, e.g. FFPE tissue (Formalin Fixed Paraffin Embedded). The sample may comprise any cell type that contains nucleic acid including all types of cells as discussed above.

A representative sample for in situ detection may comprise a fixed tissue section, a fresh frozen tissue or a cytological preparation comprising one or more cells. The sample may be permeabilized to render the nucleic acid, e.g. RNA, accessible. Appropriate means to permeabilize cells are well known in the art and include for example the use of detergents, e.g. appropriately diluted Triton X-100 solution, e.g. 0.1% Triton X-100, or Tween, 0/1% Tween, or acid treatment e.g. with 0.1M HCl. Permeabilization of tissue samples may also comprise treatment of the sample with one or more enzymes, e.g. pepsin, proteinase K, trypsinogen or pronase, etc. Also, microwave treatment of the sample may be carried out as known in the art.

The sample may also be treated to fix nucleic acid, e.g. RNA, contained in the cells to the sample, for example to fix it to the cell matrix. Such procedures are known and described in the art. For example, in the field of in situ hybridization, reagents are known for fixing mRNA to cells. In particular, 5′ phosphate groups in the RNA may be linked to amines present on proteins in the cellular matrix via EDC-mediated conjugation (EDC: 1-ethyl-3-(3-dimethylaminopropyl) carbodiimide), thus helping to maintain the localization of the RNA relative to other cellular components. Such a technique has previously been described in relation to microRNAs and their detection via in situ hybridization. Alternatively, in procedures where a reverse transcription step is performed to generate cDNA from a RNA target, the 5′ end of the cDNA primer and/or cDNA molecules can be subject to crosslinking activity. This may be achieved using for example DSP chemical or Acrylic acid NHS ester.

Since the target nucleic acid molecule need not itself be the target analyte of the assay, but can for example be a reporter molecule used or generated in the course of an assay for any desired analyte, the sample need not be a sample which naturally contains nucleic acid, or a source of nucleic acid (e.g. a cell or virus, or biological or clinical material etc.). As indicated above, the sample may be a synthetic or artificial sample. It may accordingly be a sample which has been subjected to a detection assay for an analyte in which a target nucleic has been generated, or to which a target nucleic acid molecule has been added. It may be a reaction mixture, or a reaction product, for example the product resulting from an immunoassay to detect a target analyte, e.g. an immunoPCR, immunoRCA, or proximity assay (e.g. proximity ligation assay (PLA) or proximity extension assay (PEA), as discussed above.

The target analyte may be any analyte it is desired to detect. As discussed above, in embodiments the target nucleic acid molecule of the method herein is the target analyte. In other embodiments, where the target nucleic acid molecule is a reporter, the target analyte may be any analyte it is desired to detect. The analyte may be a nucleic acid, a protein (which term includes peptides and polypeptides), or any other chemical or biological molecule or moiety, including for example carbohydrates, e.g. such as may occur as glycosyl groups on proteins. The target analyte may thus be a modified protein, for example with a post-translational modification which is detected in an assay for an analyte.

In an embodiment, the target analyte may be a protein or component of a proteinaceous molecule which is detected on the surface of a cell, or vesicle, or other cellular or sub-cellular compartment. For example, extracellular vesicles, or exosomes, may be detected and distinguished by virtue of different proteins present on their surface. Prostasomes have been proposed as biomarkers for prostate cancer, and a particular or selected prostasome or other extracellular vesicle may be detected and distinguished by detecting one or more surface proteins thereon.

The padlock probes may comprise one or more further sequences which may serve to introduce a sequence into the ligated product, and thereby into the RCP (as a complementary copy. This may be, for example, a tag or detection sequence, e.g. a barcode or identificatory motif, or a binding site for a detection probe or primer. This is particularly, the case for the second padlock probe. Such a further sequence may be found, for example, in a portion of the backbone region of the padlock probe, that is the region between the target-binding regions. In a dumbbell probe it may be in the the duplex region of the probe. Tags such as barcodes or probe/primer binding sites may be designed with different needs/purposes, for example, to introduce a universal or common sequence to enable different ligated probes in a multiplex setting to be processed together, e.g. to introduce a binding site for a universal or common amplification primer. This would enable different ligated probes to be amplified together, e.g. in a library amplification by PCR or RCA.

In particular, the second padlock probe may contain a detection sequence by which it may be detected. A complement of the detection sequence will become incorporated into the second RCP, and may be detected, for example by the binding to it of a detection probe, or by sequencing. The detection sequence may be specific to the padlock probe, and thus to the target sequence, or sequence variant it is desired to detect. Thus, each second padlock probe may have a different detection sequence. The detection sequence may be detected to detect or identify which second padlock probe was amplified in the RCA, and hence target sequence was present. Such a protocol may be applied, for instance, in the context of the method for detecting a target sequence variant, where each second padlock is provided with a detection sequence specific to particular variant. The detection sequence may thus be seen as a marker or identification sequence. The term “detection sequence” as used herein includes both the detection sequence as it occurs in the padlock probe, and the complementary copy as it appears in the RCP.

Accordingly, a tag/barcode sequence, including particularly a detection sequence, may be used to “label” different padlock probes so that they, or their ligation or amplification products may readily be distinguished from one another. Additionally, such a sequence may be used to tag different samples etc. for example so that they may be pooled (i.e. a “sample” tag or marker). Thus, in a multiplex setting, different probes (i.e. probes for different target nucleic acid sequences or different variants) may be provided with different tag sequences (e.g. different marker or detection sequences) and/or they may be provided with the same tag sequence(s) e.g. for the introduction of a common or universal sequence. Such methods may be used in conjunction with particular detection methods, including the use of detection probes or sequencing methods such as sequencing by hybridization, sequencing by ligation or other next generation sequencing chemistries, e.g. in the multiplexed detection of multiple target nucleic acids in a sample.

The term “hybridisation” or “hybridises” as used herein refers to the formation of a duplex between nucleotide sequences which are sufficiently complementary to form duplexes via Watson-Crick base pairing, or any analogous base-pair interactions. Two nucleotide sequences are “complementary” to one another when those molecules share base pair organization homology. Hence, a region of complementarity in a molecule or probe or sequence refers to a portion of that molecule or probe or sequence that is capable of forming a duplex. Hybridisation does not require 100% complementarity between the sequences, and hence regions of complementarity to one another do not require the sequences to be fully complementary, although this is not excluded. Thus, the regions of complementarity may contain one or more mismatches. Accordingly, “complementary”, as used herein, means “functionally complementary”, i.e. a level of complementarity sufficient to mediate a productive hybridisation, which encompasses degrees of complementarity less than 100%. The degree of mismatch tolerated can be controlled by suitable adjustment of the hybridisation conditions. Those skilled in the art of nucleic acid technology can determine duplex stability empirically considering a number of variables including, for example, the length and base pair composition of the respective molecules or probe oligonucleotides, ionic strength, and incidence of mismatched base pairs, following the guidance provided by the art. Thus, the design of appropriate probes, and binding regions thereof, and the conditions under which they hybridise to their respective targets is well within the routine skill of the person skilled in the art.

A region of complementarity, such as for example to a target sequence in the binding region of a padlock probe, or between a detection sequence and a detection probe, or a RCA primer to the circularised padlock probe etc., may be at least 6 nucleotides long, to ensure specificity of binding, or more particularly at least 7, 8, 9 or 10 nucleotides long. The upper limit of length of the region is not critical, but may for example be up to 50, 40, 35, 30, 25, 20 or 15 nucleotides. A complementary region may thus have a length in a range between any one of the lower length limits and upper length limits set out above. In the case of a padlock probe, the length of an individual target-binding region may be in the lower ranges, so that the total length of the two binding regions when hybridised to their target is within the upper ranges. For example an individual target binding region may be 8-15, e.g. 10-12 nucleotides, so that the total hybridised length is 16-30 nucleotides long, e.g. 20-24. It may be desirable, within the constraints of conformation of the probes, and spacing of the domains, and desired or favoured hybridisations, to minimise the total length of a padlock probe to minimise the size of the circle which is subjected to RCA, and hence to minimise the lengths of the complementary regions where possible.

The second RCP (and/or any further generation RCP) may be detected using any convenient protocol. Depending on the target sequence to be detected, the purpose of the method, and/or the specific details of the procedures employed in the method, the detection protocol employed may detect the RCP non-specifically or specifically.

For instance, the RCP may be detected directly, e.g. the concatemer may be cleaved to generate monomers which may be detected using gel electrophoresis, or more typically by hybridizing labelled detection probes that hybridize to the detection sequence in the RCP, as discussed above. The latter may particularly be used in in situ methods. The detection probe need not, however, be directly labelled. For example, the detection probe may be an unlabelled probe which functions as a sandwich probe. The concept of sandwich probes is well known in the art and may be applied according to any convenient protocol. The sandwich probes can bind to the RCP but are not directly labelled themselves; instead, they comprise a sequence to which labelled secondary oligonucleotides can bind, thus forming a “sandwich” between the RCP and the labelled secondary oligonucleotide.

A RCP may also be detected using non-sequence-specific nucleic acid labelling methods, e.g. DNA binding stains or dyes, which are widely known in the literature, or by using labelled nucleotides for incorporation into the RCP. Alternatively, the RCP may be detected indirectly, e.g. the product may be amplified by PCR and the amplification products may be detected.

The RCP may be detected using any of the well-established methods for analysis of nucleic acid molecules known from the literature including liquid chromatography, electrophoresis, mass spectrometry, including CyTOF, microscopy, real-time PCR, fluorescent probes, microarray, colorimetric analysis such as ELISA, flow cytometry, mass spectrometry, or by turbidometric, magnetic, particle counting, electric, surface sensing, weight-based detection techniques. Generally speaking, such techniques are relevant for in solution assays.

For a localised in situ detection method, the RCA product may generally be detected using labelled detection probes, which may be labelled with any detectable label, which may be directly or indirectly signal-giving. For example, the label may be spectroscopically or microscopically detectable, e.g. it may be a fluorescent or colorimetric label, a particle or an enzymatic label. Any of the labels used in immunohistochemical techniques may be used.

In multiplex procedures for detecting different target sequences and/or variant target sequences different second RCPs products may be detected and distinguished by in situ sequencing, including for example sequencing by synthesis, sequencing-by-hybridisation and sequencing by ligation, next generation sequencing and/or sequential barcode decoding techniques, including by sequencing-by-synthesis, -ligation or -hybridisation, and/or by using detection probes. Depending on the level of multiplexing, combinatorial labelling methods may be used, according to techniques well known in the art. For example, the large number of repeated sequences in the sRCA (SafeLock) products can enable distinction amongst large numbers of such products via ratio labelling with fluorescent or other spectrophotometrically detectable probes. Such ratio-labelled detection probes may be used during flow cytometry, or microscopic detection techniques, e.g. imaging, to detect large numbers of sequences, e.g. the combination of at least two fluorophores at different ratios can lead to generation of multiple populations of fluorescent labels. For example, it has been found that using combinations of two fluorophores at different ratios 7 different populations can be created. This may be expanded using 3- or 4-colour combinations.

In methods which involve the use of detection probes, the detection probe or any secondary labelling probe may be labelled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In many embodiments, the label is a directly detectable label, where directly detectable labels of interest include, but are not limited to: fluorescent labels, radioisotopic labels, chemiluminescent labels, and the like. In many embodiments, the label is a fluorescent label, where the labelling reagent employed in such embodiments is a fluorescently tagged nucleotide(s), e.g. fluorescently tagged CTP (such as Cy3-CTP, Cy5-CTP) etc. Fluorescent moieties which may be used to tag nucleotides for producing labelled probe nucleic acids (i.e. detection probes) include, but are not limited to: fluorescein, the cyanine dyes, such as Cy3, Cy5, Alexa 555, Bodipy 630/650, and the like. Other labels, such as those described above, may also be employed as are known in the art.

Although various detection modalities may be employed, conveniently the second RCPs may be detected by microscopy or flow cytometry. In both cases directly or indirectly labelled detection probes may be used, for example with fluorescent labels which may readily be detected. In particular, in a microscopy-based method, the RCPs may be detected by imaging, as shown in the Examples below. The use of such detection techniques advantageously allow the second RCPs to be digitally recorded. Indeed, since the degree of signal amplification afforded by the present method allows the second RCPs to be visualised, they may be detected by a camera or any device including a camera, such as a mobile phone.

To detect second RCPs generated in a homogenous format, they may be captured or brought down to a solid support, or surface, to facilitate imaging, or microscopic detection more generally. A second RCP, being a second generation RCA product, is larger and heavier and hence readily amenable to bringing down to a surface by centrifugation. Thus, for example, plates may readily be spun to bring second RCPs down to the bottom of a well for detection by microscopy, and particularly imaging.

As noted above, also provided herein are kits for performing the methods. The kits may include the padlock probes as discussed above, optionally together with one or more reagents and/or instructions for use of the kit. Such reagents include dNTPs, and polymerase and ligase enzymes, as well as RCA primers for the first and/or second RCA reactions. Further, components may include buffers or other reaction components for one or more of the various reactions. Still further optional components may include means or reagents for detecting the second RCP. This may include for example, detection probes and any necessary secondary labelling reagents, including for example as discussed above. Further optional components may include a solid support and/or means for capture and/or immobilisation of a target nucleic acid molecule, or of a reaction component. Instructions may be for example in printed form, or on a computer-readable medium, or as a website address.

As noted above, the method may be performed using a solid phase, for example, in which the first RCA product becomes immobilised on a solid phase, permitting the use of washing steps. This may result from immobilisation of the target molecule, for example in in situ detection procedures. The use of solid phase assays offers advantages, particularly for the detection of difficult samples: washing steps can assist in the removal of unbound and/or unligated probes etc., inhibiting components, and targets or analytes can be enriched from an undesirably large sample volume.

Immobilisation of the first RCA product and/or target molecule on a solid phase may be achieved in various ways. Accordingly, several embodiments of solid phase assays are contemplated. In one such embodiment, the sample may be provided on a solid support, such an in an in situ application for example. Alternatively, the target nucleic acid molecule may be captured by an immobilised (or immobilisable) capture probe, and the first RCA product can be generated such that it is attached to the analyte, for example by virtue of the primer for the first RCA being the target molecule or being attached to the target molecule. Alternatively, the first RCA product may simply be immobilised to a solid support. For example, the primer for the first RCA may be provided with an immobilisable group or moiety or means for immobilisation, or may be immobilised, prior to the first RCA.

The manner or means of immobilisation and the solid support may be selected, according to choice, from any number of immobilisation means and solid supports as are widely known in the art and described in the literature. Thus, the capture probe, or first RCA primer or first RCA product may be directly bound to the support (e.g. chemically crosslinked), it may be bound indirectly by means of a linker group, or by an intermediary binding group(s) (e.g. by means of a biotin-streptavidin interaction). Thus, a capture probe or first RCA primer or first RCA product may be provided with means for immobilisation (e.g. an affinity binding partner, e.g. biotin or a hapten or a nucleic acid molecule, capable of binding to its binding partner, i.e. a cognate binding partner, e.g. streptavidin or an antibody or a nucleic acid molecule) provided on the support. A capture probe may be immobilised before or after binding to the target molecule. Further, such an “immobilisable” capture probe may be contacted with the sample together with the support. Analogously, a first RCA primer may be immobilised before or after the first RCA etc.

The capture probe may be, for example, an antibody or nucleic acid molecule that is capable of binding to the target molecule specifically.

The solid support may be any of the well-known supports or matrices which are currently widely used or proposed for immobilisation, separation etc. These may take the form of particles (e.g. beads which may be magnetic or non-magnetic), sheets, gels, filters, membranes, fibres, capillaries, or microtitre strips, tubes, plates or wells etc.

The support may be made of glass, silica, latex or a polymeric material. Suitable are materials presenting a high surface area for binding of the analyte. Such supports may have an irregular surface and may be for example porous or particulate e.g. particles, fibres, webs, sinters or sieves. Particulate materials e.g. beads are useful due to their greater binding capacity, particularly polymeric beads. Conveniently, a particulate solid support may comprise spherical beads. Monodisperse particles, that is those which are substantially uniform in size (e.g. size having a diameter standard deviation of less than 5%) have the advantage that they provide very uniform reproducibility of reaction.

As noted above, the target nucleic acid may itself may be immobilised (or immobilisable) on the solid phase e.g. by non-specific absorption. In a particular such embodiment, the molecule may be present within cells, being optionally fixed and/or permeabilised, which are (capable of being) attached to a solid support, e.g. a tissue sample comprising the target molecule may be immobilised on a microscope slide.

Advantages of the methods herein are discussed above. Such advantages are particularly beneficial in the context of detecting a target sequence or variant in complex samples, or where they are present in low abundance. As discussed above, the present method provides a high degree of signal amplification, rendering the method very sensitive. The method is thus particularly suited to detecting or identifying very rare sequence variants. The method can be used, for example, to find and detect tumour-derived mutant DNA sequence in patient samples, including notably cell-free DNA in plasma. The method may thus find utility in the diagnosis or monitoring of cancer, or e.g. to reveal recurrence of the cancer. The method may be used in the context of any cell-free DNA, and may also find application in prenatal testing including particularly NIPT. The technique is rapid, has minimal instrument requirements, and allows multiplex analysis of sequence variants for enhanced sensitivity.

The detection of low frequency or rare sequence variants or mutations also requires high specificity of detection, and a minimised risk of introducing artificial sequence alterations that could be mistaken for variant sequences This is also afforded by the combination in the present method of the use of a gap-fill padlock mechanism and RCA amplification, and the targeting of the padlock in-fill by a second padlock probe.

In the present method, the first RCP is generated in a highly specific manner, and the generation of the second RCP at high amplification is dependent on the presence of the first RCP.

Each RCA product typically contains several hundreds of, or in some cases in the order of thousands of, complements of the RCA template circle (e.g. circularised padlock probe) that was used to generate it. The second generation RCA product, which is obtained by RCA of the circularised second padlock probes on the first RCA product, therefore can contain, for example, 1000×1000 monomer sequences, and is thus of a large size, and can have molecular weights which reach tens of gigaDaltons. Further, as noted above, further copies of monomer sequences may be generated by further generations of RCA reactions. Such reaction products can readily be detected. The dimensions of a second RCA product can reach several micrometers, and they may readily be visualised as individual products, e.g. by microscopy. Each second, or further, RCA product can be detected as a clonal product, generated from a single target nucleic acid molecule, without the need for compartmentalisation of the RCA reactions. For example, when labelled with fluorescent detection probes, prominent brightly fluorescent reaction products allow counting and distinction of individual amplification products of single molecules across wide fields of view at low magnification (e.g. 20×). The second or further RCA reaction products are sufficiently large and bright to be recorded by standard flow cytometry. This allows ready counting, and digital scoring in a matter of minutes using generally available instrumentation, thus offering excellent quantitative precision over wide dynamic ranges. As mentioned above, the repeat sequences present in the concatemeric second or further RCA product allow analysis of products labelled with distinct combinations of fluorophores, by ratio-labelling techniques, thereby allowing increased multiplexing. Because of their large size and considerable molecular weight, sRCA (SafeLock) products may also be enriched by centrifugation in an ordinary desk-top centrifuge or similar. Indeed, lower speed centrifugation or unit gravity may suffice. The new method herein thus enables multiple advantages.

The strong signal amplification afforded by the second RCA reaction allows the ready and easy visualisation of signal, as discussed above, for example microscopically at low magnification or on a digitally scanned image and hence may permit rapid and easy visual inspection of assay results in a clinical scenario, e.g. inspection of pathology results in routine use. Thus, the methods of the invention are particularly suited to clinical analysis procedures.

The methods can be helpful to identify rare integrated copies of viral genomes in human tissues or for otherwise detecting rare RCA products such as upon inefficient mutation detection in tissues. Another example when easy identification of a rare event may be helpful is when screening for the presence of circulating tumour cells (CTC) among a vast majority of non-CTC cells. The strong signal produced by the method allows fast and easy identification of events (detection of CTCs) at low magnification.

The methods allow the detection assay to be speeded up, which may be of value in at point of care locations such as doctor's offices etc. In this regard, the second RCA can be performed in a relatively short time.

The increases in signal strength/speed may allow other means of detection beyond the conventional fluorescence based methods, for example using turbidometric, magnetic, particle counting, electric, surface sensing, and weight-based detection techniques. For example one individual sRCA product from a second generation RCA after a 1 hour amplification has the potential weight of several femtograms. Such a weight increase may be detected by methods and means known in the art such as cantilevers, surface plasmon methods, and microbalances e.g. quartz crystal microbalances etc. Further, as noted above, the increased size and weight of the second RCP allows it efficiently to be localised to a surface by centrifugation. Conveniently, this may be performed at 3000× in 15 minutes, in contrast to first generation RCA products, which cannot efficiently be captured by centrifugation using a bench-top centriguge.

The present method can enable the generation of an enhanced signal which is localised to the product of the first RCA, it also confers the ability to count individual reaction products (second RCA products) using standard flow cytometers or distributed on a planar surface, etc. for highly precise digital detection. In particular the second RCPs may be stained with chromogenic reagents such as HRP, and imaged via a smart phone camera. Thus, the method may permit an equivalent reaction to digital PCR, but with no need for emulsions or microfabricated structures, or finding conditions where exactly one template is present per compartment.

The prominent amplification products derived from the method of the present invention will further permit cloning of individual RCPs, since the product obtained from an individual first RCA template may be visualised. An individual second RCA product may therefore be identified and isolated. For example, with the aid of the amplification method of the present invention visualization can be achieved in low melt agarose for isolation with no need for magnification, and the product may then be isolated e.g. scoped out with a toothpick, analogously to the isolation of bacterial colonies.

The detection of rare mutations can be very important clinically for diagnosis. For example mutations in certain genes (e.g. KRAS mutations) can be diagnostically important and may serve to identify the emergence of acquired resistance to particular therapies (e.g. anti-EGFR therapy). Much effort has focused in recent years on developing methods for detecting such mutations. The method of the present invention could provide a useful addition to such methods.

In addition to enabling the detection of point mutations present at low frequencies in DNA samples, the present method also provides a powerful means of screening DNA samples for the presence of any and all of a very large number of distinct target sequences in a manner that is not possible by PCR or any other current method. The target selectivity achieved by the present method, which requires target recognition by the two ends of the gap fill padlock probes, is similar to that of PCR with its pairs of primers. However, unlike in PCR, the intramolecular reactions of the gap fill padlock probes allow for several hundred thousands of probes to be applied in parallel with no deterioration of target selectivity.

In this method, a second padlock reaction targeting the filled-in sequence has the effect of further increasing detection specificity as any spurious reaction products from the first gap fill padlocks would not be recognised by the secondary padlock probes, whether gap-fill or not. This is analogous to the use of nested PCR to increase detection specificity.

After each probe circularisation reaction, remaining unreacted probes and sample nucleic acid sequences can be removed using mixes of exonuclease that spare the successfully circularised probes.

Furthermore, the discrete nature of the sRCA products allows digital detection by production of one reaction product for each detected target molecule and collection of these prominent reaction products, with minimal risk for mix-up with any other material in the reaction. For example, a probe mix could be created for all types of bacteria or all species of insects or fungi. This could then be used to identify positive reaction products, for example by amplifying tag sequences on the secondary padlock probes by PCR, and hybridising the products to tag arrays or similar.

Still further, as noted above where second, and optionally further, ligation and RCA reactions are performed using gap-fill padlocks, then a preparative production of copies of the target nucleic acid sequence becomes possible.

The present sRCA (SafeLock) method also increases the precision of genotyping by interrogation of the repeated sequences of individual RCA products rather than of individual target sequences. Thereby, occasional mistypings by padlock probes can be tolerated without resulting in erroneous results as long as they are considerably rarer than the correct results within an individual RCA product. This allows for genotyping via a majority-vote mechanism. This can also have consequences for how the padlock probe-based genotyping is done, where it may be possible to enhance sequence distinction by using conditions where neither variant is detected with 100% efficiency, as long as the ratio between correct and incorrect reactions is satisfactory, and a sufficient number of repeats are detected by the padlock probes.

The method will now be described in more detail with reference to the following Figures and non-limiting examples.

### DESCRIPTION OF FIGURES

FIG. 1: schematic illustration of the method. The gap-fill-ligation, first RCA, second padlock (referred to as the genotyping padlock) ligation, and second RCA steps are shown. The second RCP is detected by differentially-labelled detection probes.

FIG. 2: demonstration of gap-fill over extension. The conventional padlock ligation is one condition of the gap-fill-ligation probe where the gap is zero. Lane 1 shows 10 nM padlock/10 nM ligation template (ligtemp)+Ampligase with dNTP; lane 2 shows 110 nM padlock/10 nM ligation template (ligtemp)+Ampligase=polymerase with dNTP; lane 3 shows 10 nM padlock/10 nM ligation template (ligtemp) control. When lanes 1, 2 and 3 were cooling down from denaturing temperature to the annealing temperature in the absence of polymerase, intact circles were efficiently formed (see lane 3 for no ligase control). However, when the polymerase was present during the denaturing->annealing steps, over extended products were formed (lane 2). dNTPs were present in both the reactions loaded in lanes 1 and 2.

FIG. 3: Single cycle one-step gap-fill-ligation protocol investigation. In this experiment, 50 ng gDNA is used. The Annealing temperature and ramping speed as well as the effect of crowding reagent is investigated.

FIG. 4: 50 ng genomic DNA was used in cycling gap-fill-ligation reactions for the indicated numbers of cycles. A second RCA was performed to produce easily detectable sRCA products that were enumerated by microscopy and using image processing algorithms.

FIG. 5: The effect of a crowding agent—PEG—was evaluated in the cycled gap-fill-ligation protocol after 4 reaction cycles.

FIG. 6 Image data from cycled gap-fill protocol in combination with sRCA protocol to detect genomic DNA spike in samples, KRAS G12D genomic DNA were spiked-in wild-type genomes at 50 ng gDNA levels. The images show wild-type (WT) (top) and mutant (G12D) (bottom) image channels separately.

FIG. 7: Quantification of products from the serial dilution experiment illustrated in FIG. 7, after allele-specific detected via sRCA protocol. Two replicates were performed, and each data point was calculated on the basis of four different data images acquired in the same reaction at random positions.

FIG. 8: 50 ng wild-type gDNA samples were analyzed with gap-fill-ligation sRCA protocol. KRAS Wild-type and KRAS G12S second RCA probes were used to detect the gap-filled circles and they give rise to second RCA products in their respective channel indicated in the figure. The images show wild-type (WT) (top) and mutant (G12S) (bottom) image channels separately. Signal detected from the Mutant channel, the rectangle over each sequence indicates the gap size and the triangle indicates the target base that the genotyping probes are detecting.

FIG. 9: KRAS detection targeting cellular RNA molecules in situ. A549 cell line is homozygous for the KRAS G12S mutation. When both wild-type and KRAS G12S sRCA-specific probes were applied the resulting sRCA products were detected with separate colours, but no combination of the two colours was seen. The nuclei, WT, and mutant (G12S) channels are shown separately, from top to bottom respectively.

FIG. 10: KRAS detection targeting cellular RNA molecules in situ. The HaCAT cell line is homozygous for the wild-type KRAS gene. When both wild-type and KRAS G12S sRCA detection probes were applied to detect the gap-filled products the sRCA products were consistently red, demonstrating the presence of wild-type KRAS. The nuclei, WT, and mutant (G12S) channels are shown separately, from top to bottom respectively.

FIG. 11: KRAS detection targeting cellular RNA molecules in situ. The HaCAT cell line is homozygous for wild-type KRAS while the A549 cell line is homozygous for the mutant KRAS G12S allele. Cells from the two cell lines were co-cultured on the same slides at a ratio of 100 HaCAT for every A549 cell. We applied the gap-fill-ligation sRCA protocol using wild-type KRAS probe and KRAS G12S sRCA probes to detect the RNA-derived gap-fill-ligation products in respective cell line. The data were recorded under an 10× objective lens. The DAPI, WT for HaCaT cell line, and mutant (G12S) for A549 cell line channels are shown separately, from top to bottom respectively.

FIG. 12: SafeLock RNA detection of DNMT3A in lymphoma tissue samples. FIG. 12A shows DNMT3A RNA detection using DNMT3A-MUT (I66T+E733G) probes and DNMT3A wild-type probes on a sample from patient HI185-13, who is heterozygous for the I66T and E733G mutations in the DNMT3A gene. FIG. 12B shows DNMT3A RNA detection using DNMT3A-MUT (I66T+E733G) probes and DNMT3A wild-type probes on a sample from patient A23066-10, who is homozygous for the DNMT3A wild-type gene. The gap-fill-ligation sRCA protocol was applied using DNMT3A-MUT (I66T+E733G) and DNMT3A wild-type probes to detect the RNA-derived gap-fill-ligation products in the samples. The data were recorded under a 40× objective lens. The combined DNMT3A-MUT (I66T+E733G) mutant and DNMT3A wild-type channels are shown on the left in the micrographs from both patient samples, and the separate channels for the probes are shown in the center and on the right respectively.

### EXAMPLE

### Materials and Methods for Solution Phase Protocol

### Extraction of Cell-Free Plasma DNA

DNA extraction from plasma was done using the QIAamp Circulating Nucleic Acid kit (Qiagen cat. 55114). DNA was extracted from up to 2 mL plasma from each patient and eluted in 50 μL elution buffer.

### Gap-Fill Cycling Based Probing and Amplification

Sequences of interest in cell-free plasma DNA were incorporated into gap-fill probes (“first padlock probes) in the cycling fashion with following mixture: in 20 μl reaction containing 1× phi29 buffer, 0.25 μM dNTP, 100 nM gap-fill probes, 1 mM NAD, 0.5 U/μl Ampligase and 0.0025 U/μl Stoffel DNA polymerase, 2.5 ng/μl cfDNA sample and 15% PEG4000. The cycling incubation program was as follows: 95° C. for 60 sec, 10 cycles of 95° C. for 60 sec, 60° C. for 120 sec, 32° C. for 120 sec, 45° C. for 300sec.

Exonuclease clean-up. 10 μL of clean-up mixture contains 0.5 U/μl Exonuclease I(NEB), 2.5 U/μl Exonuclease III(NEB) and 0.1 U/μl Exonuclease Lambda (NEB) were mixed with 20 μl gap-fill products. The mixtures were incubated at 37° C. for 60 min, followed by 85° C. for 20 min.

Target sequence amplification by a first RCA. Gap-filled circular products (ligated first padlock probes) containing target nucleotides were amplified through rolling circle amplification. 5 μl of RCA buffer containing 1× Phi29 buffer, 700 nM Primer, 1.4 mM dNTP (Thermo Scientific) and 1 U/μl Phi29 polymerase was added to the reaction mixture. The reactions were incubated at 37° C. for 30 min, then 65° C. for 10min.

Genotyping of RCA products via second padlock probe ligation. Genotyping padlock probes second padlock probes) were hybridized to first generation RCA products (first RCPs) and ligated in a sequence-specific manner, by adding 5 μL ligation mix containing 1×Phi29 buffer, 3 mM NAD, 0.6 U/μl Ampligase, and 100 nM genotyping padlock probe pairs to the reaction mixtures and incubating at 45° C. for 30 min.

Second RCA—sRCA. Ligated genotyping (second) padlock probes, encircling the first generation RCA products, were amplified in a secondary RCA reaction. 30 μL RCA mixture containing 1×Phi29 buffer, and 1.2 U/μl Phi29 DNA polymerase was added to the genotyping ligation mixtures and incubated at 37° C. for 10 min. Then 5 μL of the reaction mixture containing 2.4 mM dNTP (Thermo Scientific), and 1.3 μM primers was added to the reaction mixtures, and the reactions were incubated at 37° C. for 30 min.

Digital recording of sRCA products (second RCA products) by flow cytometry. The final reaction mixtures containing sRCA products were diluted into hybridization buffer containing 100 nM fluorophore-labeled oligonucleotide probes, specific for the different sRCA products, in 20 mM Tris-HCl, 20 mM EDTA, 0.1% Tween 20 and 1M NaCl to a final volume of 250 μl. The solution was applied onto the Fortessa flow cytometer (BD Bioscience) and sRCA products were counted at ‘Slow’ speed for 150 seconds per sample.

### Additional Methods

Non-cycled Gap-fill based probing and amplification. (FIG. 3 and Example 2) Sequences of interest in cell-free plasma DNA were incorporated into gap-fill probes in the non-cycling protocol with following mixture: in 20 μl reaction containing 1×phi29 buffer, 0.25 μM dNTP, 100 nM gap-fill probes, 1 mM NAD, 0.5 U/μl Ampligase and 0.0025 U/μl Stoffel DNA polymerase, 2.5 ng/μl cfDNA sample, depending on the experiment setup, 10% PEG4000 was added in half of the reactions and no PEG4000 was added into the control reactions. The incubation program was as follows: 95° C. for 60 sec, 65° C./60° C./55° C. for 600 sec (The ramping speed for this step was set to 100% or 4% on the Veriti PCR machine(Applied Biosystem)), 32° C. for 600 sec, 45° C. for 1200 sec.

### Additional Protocol for Example 8

Cell and pretreatment. Human HaCaT and human Lung cancer A549 cell lines were cultured in Dulbecco's Modified Eagles medium (Gibco) without phenol red, with 10% fetal calf serum (Sigma), 2 mM L-glutamine (Sigma). Two Cell line were cultured at 100:1 ratio on SuperFrost plus slides (Thermo Scientific) and fixed in 70% ethanol at room temperature after a wash in PBS. Permeabilization was made by using 0.01% pepsin from porcine gastric mucosa (Sigma, CAS# 9001-75-6) in 0.1MHCl for 90 s at 37° C., followed by washing in PBS and dehydration by serial incubation in ethanol (70%, 85%, 100%).

cDNA synthesis for mRNA detection. cDNA synthesis was performed in the supplied M-MuLV RT reaction buffer (50 mM Tris-HCl pH 8.3, 75 mM KCl, 3 mM MgCl2, 10 mM DTT), 0.5 mM dNTPs, 0.2 g/BSA (New England Biolabs), 0.1 μM of each primer and 10 U/I of TRANSCRIPTME M-MuLV reverse transcriptase (DNA Gdansk, Poland) for 2 h at 45° C. Slides were washed twice in PBS and then fixed in 3% paraformaldehyde for 5 min and washed twice in PBS before the hybridization of gap-fill padlock probes.

Hybridization of the Gap-fill probes. Hybridization were performed in a mixture containing 0.1 M of the padlock gap probe 1× Ampligase buffer (20 mMTris-HCl pH 8.3, 25 mM KCl, 10 mM MgCl2, 0.5 mM NAD and 0.01% Triton X-100), 0.2 g/l BSA and 100mM KCl at 45° C. for 60 min.

Gap-fill and ligation of the gap-fill padlock probes. The polymerization and ligation reaction mixture containing 1× Stoffel buffer, 10 μM dNTP, 1 mM NAD, 0.5 U/μl Ampligase and 0.0025 U/μl Stoffel DNA polymerase were added into the samples and incubated at 37° C. for 1 hour and then 45° C. for 1 hour.

Target sequence amplification by a first RCA. Gap-filled circular products (ligated first padlock probes) containing target nucleotides were amplified through rolling circle amplification. In the rolling circle amplification mixture contains 1× Phi29 buffer, 100 nM Primer, 200 μM dNTP (Thermo Scientific) and 0.1 U/μl Phi29 polymerase was added to the reaction mixture. The reactions were incubated at 37° C. for 60 min, then washed with 1×PBS-T for three times.

Genotyping of RCA products via second padlock probe ligation. Genotyping padlock probes second padlock probes) were hybridized to first generation RCA products (first RCPs) and ligated in a sequence-specific manner, by adding a ligation mix containing 1× ampligase buffer, 0.6 U/μl Ampligase, and 100 nM genotyping padlock probe pairs to the reaction mixtures and incubating at 45° C. for 60 min.

Second RCA—sRCA. Ligated genotyping (second) padlock probes, encircling the first generation RCA products, were amplified in a secondary RCA reaction. The RCA mixture containing 1×Phi29 buffer, 0.1 U/μl Phi29 DNA polymerase, 200 μM dNTP (Thermo Scientific), and 0.1 μM primers was added to the reaction mixtures, and the reactions were incubated at 37° C. for 60 min.

Digital recording of sRCA products (second RCA products) by Imaging. The final sRCA products were labeled by hybridization buffer containing 100 nM fluorophore-labeled oligonucleotide probes, specific for the different sRCA products, in 20 mM Tris-HCl, 20 mM EDTA, 0.1% Tween 20, 10 ng/mL DAPI and 1M NaCl and incubated at 37 C for 60 min. The final cell specimen were mounted in Vectashield mounting medium (Vector Laboratories) and imaged with AxioPlan2 Fluoroscent Microscopy (Ziess, Germany).

### Example 1

### Demonstration of Over-Extension in the Presence of Polymerase

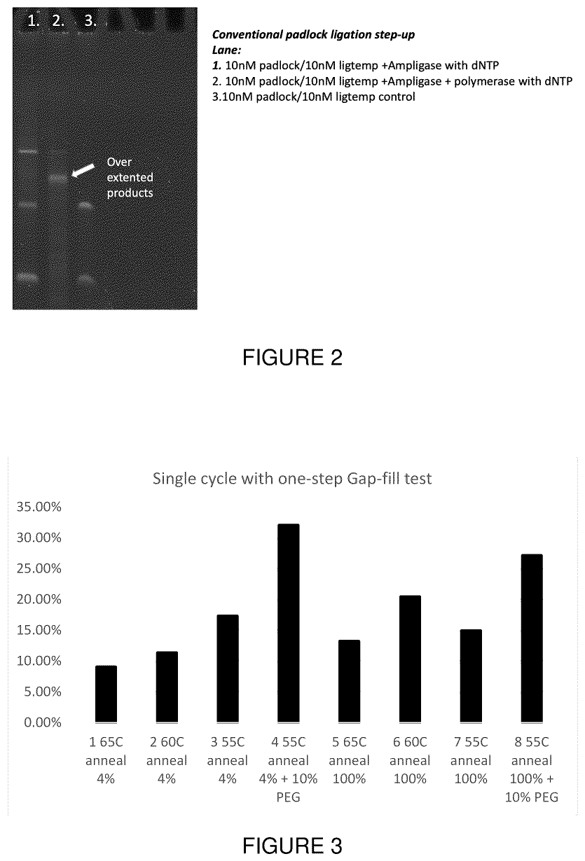

In this experiment a conventional non-gap-filling padlock probe is used to show the principle of over-extension. The padlock may be viewed as having a gap of zero. The padlocks and ligation templates are incubated in the presence of (1) ligase and dNTPS, or (2) ligase and polymerase and dNTPs. Padlocks and ligation templates alone (without ligase, polymerase or dNTPs were used as the control (3). The results are shown in FIG. 2. Lane 3 (control) shows that no ligated circles were created, as expected. When lane 1 was cooling down from denaturing temperature to the annealing temperature in the absence of polymerase, intact circles were efficiently formed (see lane 3 for no ligase control). However, when the polymerase was present during the denaturing->annealing steps, over extended products were formed (lane 2). This explains the necessity of delaying the extension reaction until after the annealing step is finished, and the 5′ end of the padlock probe is hybridized.

### Example 2

### Single Cycle One-Step Gap-Fill-Ligation Protocol Investigation

We initially investigated some conditions that might influence the gap-fill-ligation efficiency even at the single round gap-fill-ligation with one-step protocol (FIG. 3). We conclude that stable hybridization conditions as well as other conditions that influence the hybridization of probes and templates such as ramping speed and crowding reagents can be optimized to achieve a high gap-fill-ligation yield.

The results show improved yield at 55° C., in the presence of crowding agent (PEG).

### Example 3

### Demonstration of Accumulation of Ligated Padlock Probes in a Cycled Gap-Fill-Ligation Reaction

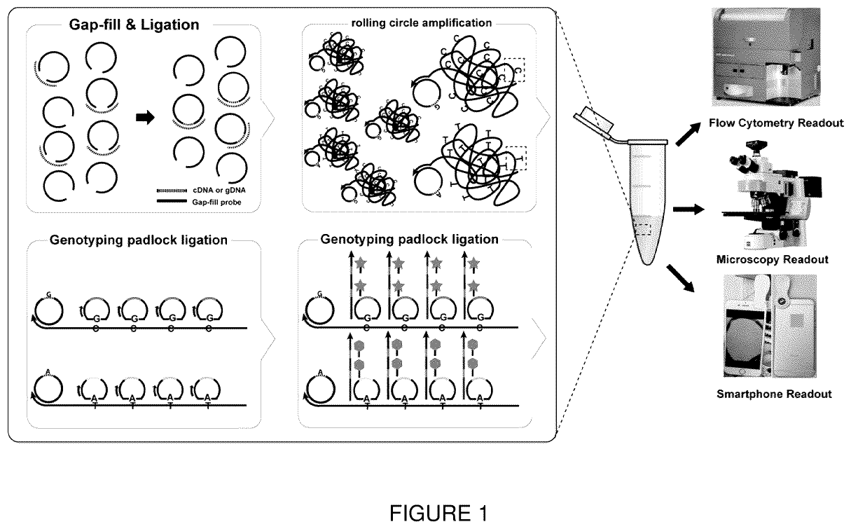

We tested the cycling-ligation gap-fill-ligation protocols using conditions determined from Example 2 on genomic DNA samples. The DNA circles that resulted were replicated by RCA, followed by genotyping using second padlock probe, and secondary RCA reaction, yielding second RCA products (sRCA products). The steps of the method are illustrated schematically in FIG. 1, and as shown in that figure they can be digitally recorded by flow cytometry, microscopy, or even a regular mobile phone camera.

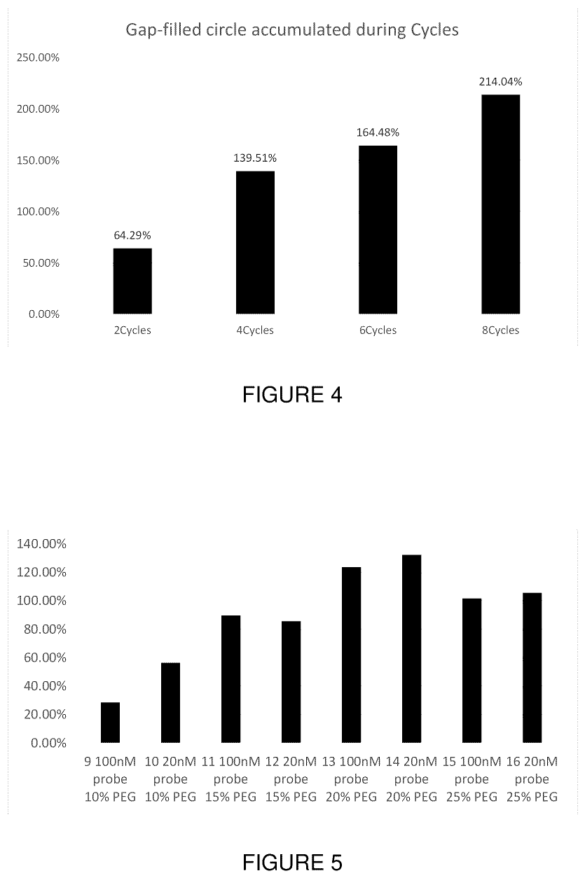

As can be seen from FIG. 4, we overserved a clear trend that the gap-filled circles accumulated with increased cycles of gap-fill-ligation, demonstrating that the cycling protocol works as intended.

### Example 5

### Effect of Crowding Agent

The crowding agent PEG was added to cycling gap-fill-ligation reactions and products formed during 4 cycles were counted after sRCA. A concentration of 20% PEG provided the optimal yield (FIG. 5).

### Example 6

### Detection of Mutants in Genomic DNA

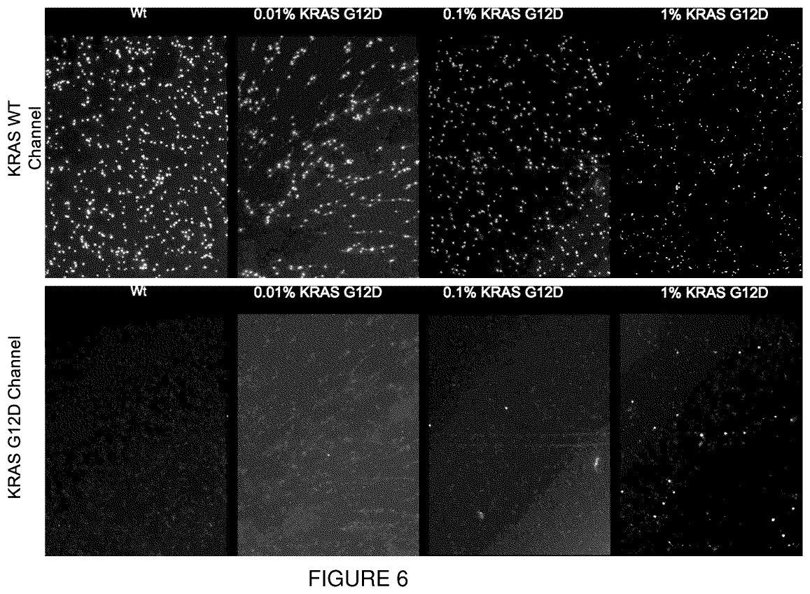

We applied the gap-fill-ligation protocol in combination with the second RCA as illustrated in FIG. 1 on genomic DNA samples where KRAS mutant genomic DNA was serially diluted into wild type genomic DNA (FIG. 6). FIG. 6 shows the WT and mutant products in separate image channels, and could be distinguished from one another.

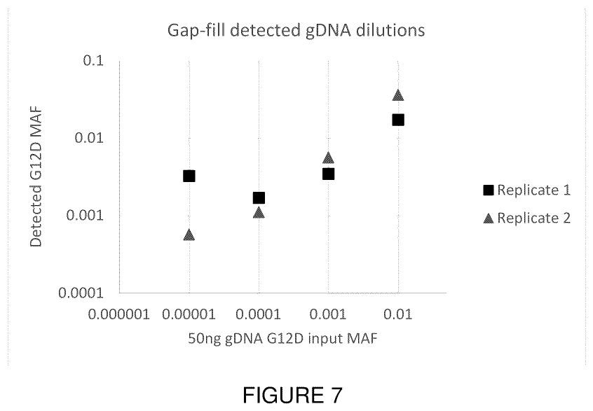

We further enumerated the sRCA (second RCA) product numbers resulting in the serial dilution experiment illustrated in FIG. 6, and the numbers are plotted in FIG. 7. The gap-fill-ligation protocol was capable of detecting KRAS G12D mutation even at a 1 in 10 000 dilution.

### Example 7

### Investigation of Gap Size and Target Mutant Position

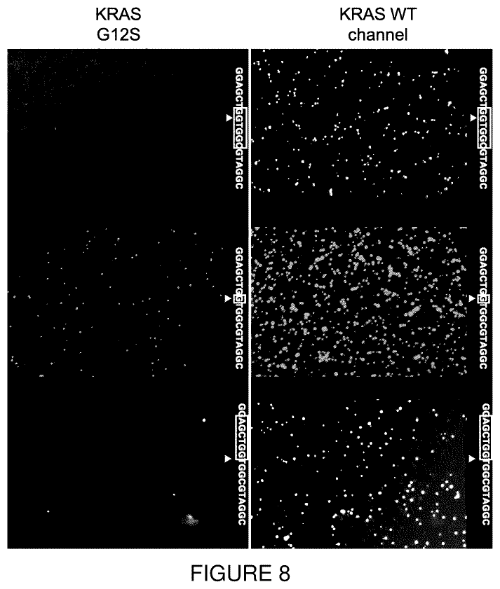

We also investigated the influence of gap size and target mutant position on the gap-fill-ligation fidelity as illustrated in FIG. 8. The sequence GGAGCTGGTGGCGTAGGC (SEQ ID NO. 5) represents the sequence in the target molecule comprising the probe binding sites and target sequence. This sequence, as shown at the top of each of the microscopy images in FIG. 8, is annotated to illustrate the gap-fill-ligation probe binding region (flanking sequences), gap-fill-ligation region (shown in rectangles) and the genotyping target base (indicated with a triangle).

The left hand panel in FIG. 8 illustrates results when a gap of 6 nt was used, and the genotyping target base was the 2^{nd} base in the gap, revealing no nonspecific products. In the right hand panel, also with a gap of 6 nt but with the genotyping target position at the last base to be filled, some nonspecific products were observed. In the middle image, the probe had a gap of a single nucleotide when hybridized to its target, with diagnostic nucleotide variant positioned in the gap. These conditions resulting in considerable numbers of erroneous products. However, the conditions may be optimized to reduce or minimize this, for example including the use of a different polymerase enzyme for the gap fill extension step. Accordingly, the gap size and also the position of the target base within the gap influence the fidelity of the gap-fill-ligation mechanism. The parameters can be optimized in a given situation. These data was generated using the Stoffel fragment, that is a variant of the Taq polymerase lacking a proof reading domain.

### Example 8

### In Situ Detection of RNA

We also applied the gap-fill-ligation/sRCA protocol to genotype RNA molecules in cells in situ, and we successfully detected the correct genotype of different cell lines at RNA level.

The sequences of the gap-fill padlock probe and target are shown in Table 1 below. The flanking sequences in the target molecule are shown underlined, the gap fill ligation region is shown in bold, and the genotyping target base is shown in double-underline.

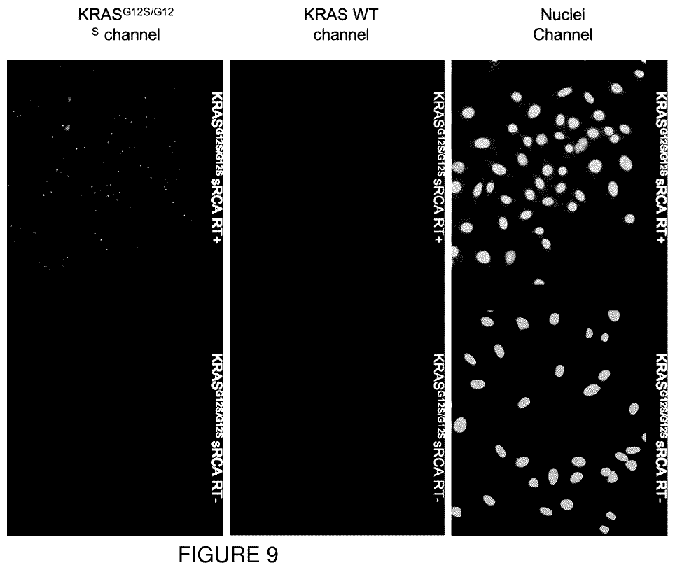

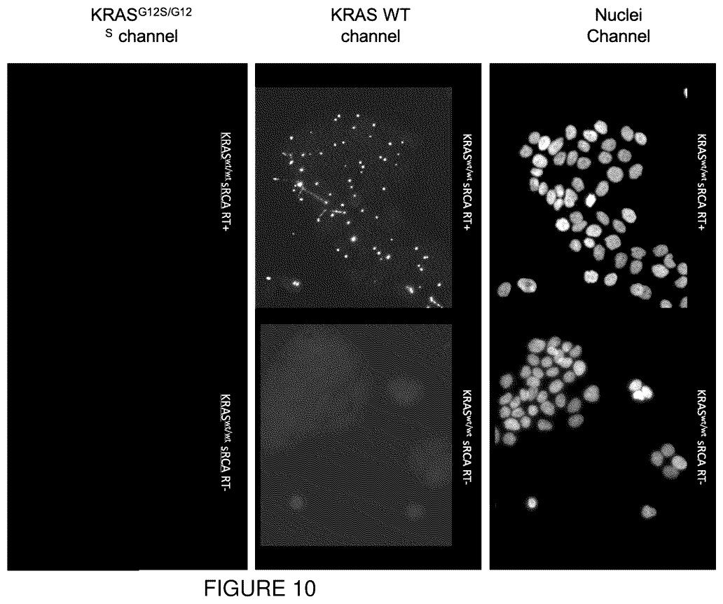

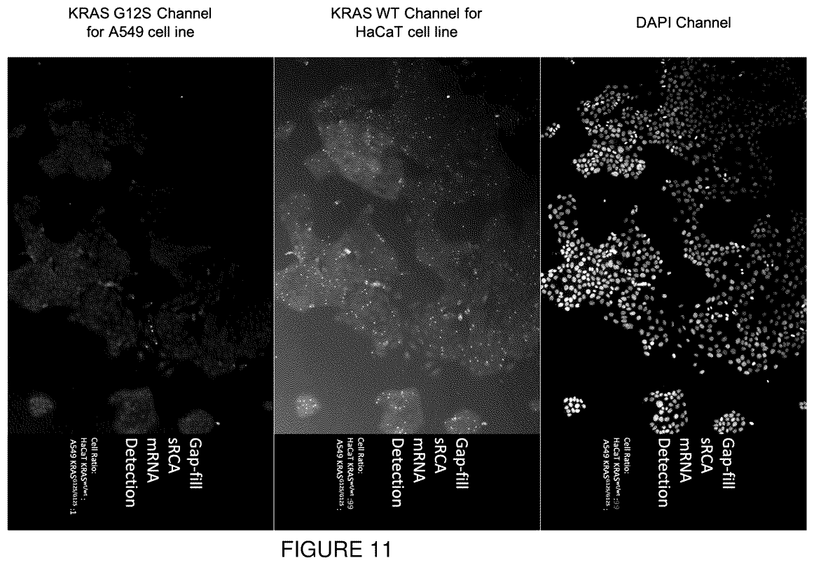

In A549 cell line samples (FIG. 9), we only detected mutant signals (as can be seen in the KRAS G12S/G12S mutant channel; no signals in the WT channel), consistent with the fact that the cell line homozygous for the KRAS G12S allele. In the absence of reverse transcriptase, no target was generated for the gap-fill-ligation probe and accordingly neither green nor red signals were detected. In HaCAT cells (FIG. 10), only wild-type signal (as can be seen in the KRAS WT channel) were detected as expected, given that this cell line is homozygous for the wild-type KRAS allele. Again, in the absence of reverse transcription enzyme, no targets are generated for the gap-fill-ligation probe, and therefore neither green nor red signals were detected. When we applied the gap-fill-ligation sRCA combined RNA detection on co-cultured A549 and HaCAT cell lines, the cells exhibited products of one or the other color, while no cells were seen to harbour two different colored sRCA products (see FIG. 11 which shows the cell lines in different channels).

### Example 9

### In Situ Detection of RNA in Fresh Frozen Tissue Samples

The gap-fill-ligation/sRCA protocol as described in the Examples above, was also applied to genotype RNA molecules in fresh frozen lymphoma tissue samples from two different patients. One patient, identified as H1185-13, carried the heterozygous DNMT3A I661T and E773G mutations and the other patient, identified as A23066-10, carried the homozygous wildtype DNMT3A gene which did not comprise said mutations, with genotype confirmed by next-generation sequencing. The correct genotype of the different tissue samples at RNA level was successfully detected by the gap-fill-ligation/sRCA protocol.

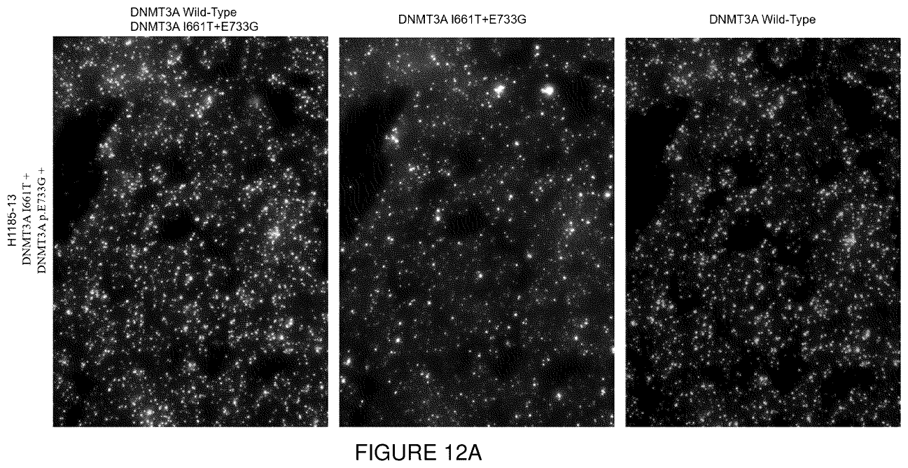

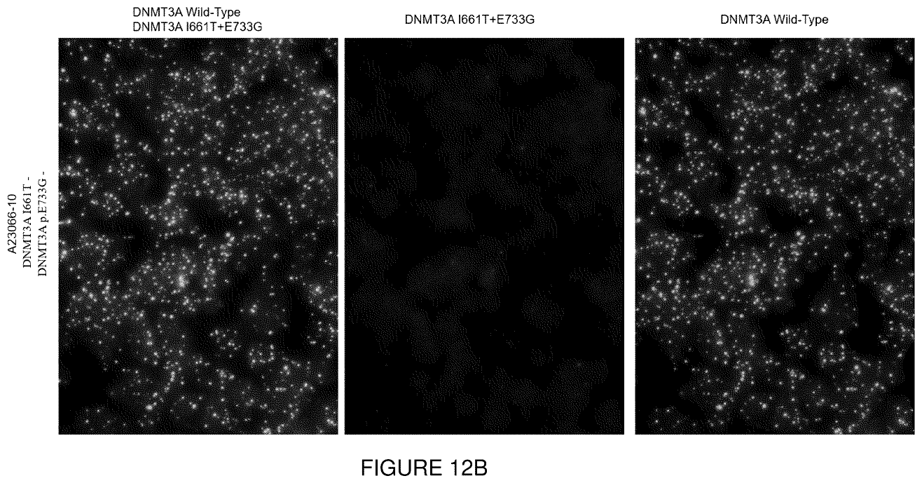

The results are respectively shown in FIG. 12 (40× magnification).

In the DNMT3A I661T+ E733G+ tissue sample (FIG. 12A), signals from both the DNMT3A-MUT (I66T+E733G) probe and DNMT3A wild-type probes were detected which is consistent with the fact that the tissue sample is derived from a patient carrying heterozygous DNMT3A I661T E733G mutations. In contrast, signals from the mutant probe were not detected from the DNMT3A I661T− E733G− tissue sample (FIG. 12B), which comprised only the wild-type copy of the gene.

TABLE 1

| | 5′arm (5′ target | Non target | 3′arm (3′ target | Gap |
|---|---|---|---|---|
| | binding region) | complementary region (backbone region) | binding region) | size |
| Gap-fill | GTAGGCAAGAGTGCCTTGACGA | TGTATCTGACTTAGCTTCCATCTTCATAGAGCGATATC | GTGGTAGTTGGAGCT | 6 bp |
| probe | TACAGCTA (SEQ ID NO. 1) | CTACGGTAGTGTATC (SEQ ID NO 2) | (SEQ ID NO. 3) | |

| | |
|---|---|
| Target | CCTGCTGAAAATGACTGAATATAAACTTGTGGTAGTTGGAGCTGGTGGCGTAGGCAAGAGTGCCTTGACGATACAGCT |
| sequences | AATTCA (SEQ ID NO.4) |

1. A method for detecting a target nucleic acid sequence in a target molecule in a sample, said method comprising:
(i) contacting the target molecule with a first padlock probe, which comprises at or near its respective 5′ and 3′ ends target-binding regions which are capable of hybridising to complementary binding sites in the target nucleic acid molecule which flank the target nucleic acid sequence, and allowing the target binding regions of the probe to hybridise to the target nucleic acid molecule;
(ii) optionally after cleavage of any unhybridised nucleotides at the 5′ and/or 3′ ends, extending the hybridised 3′ end of the padlock probe using a polymerase to create a complementary copy of the target nucleic acid sequence, and ligating the extended 3′ end to the hybridised 5′ end to circularise the padlock probe;
(iii) performing a first RCA reaction using the circularised padlock probe as a first RCA template to generate a first RCA product (RCP) comprising multiple repeats of a copy of the target nucleic acid sequence;
(iv) contacting the first RCP with a second padlock probe comprising target binding regions specific for the target nucleic acid sequence and allowing the probe to hybridise to the target sequence in the multiple repeats;
(v) ligating the hybridised second padlock probes to circularise the hybridised padlock probe;
(vi) performing a second RCA reaction using the circularised second padlock probes as a second RCA template to generate a second RCP containing multiple repeat complementary copies of the second padlock probe;
(vii) detecting the second RCP to detect the second padlock probe, and thereby the target nucleic acid sequence.

2. The method of claim 1, wherein in step (i), the 3′ target binding region of the padlock probe is at least 6 bases shorter than the 5′ target binding region and the padlock probe is contacted with the target nucleic acid molecule together with dNTPs, a polymerase and a ligase, and wherein the dNTPs are provided at a concentration of no more than 10 μM and the polymerase is provided at a concentration of no more than 0.025 U/u.

3. The method of claim 1, wherein step (i) comprises:
(a) contacting the target nucleic acid molecule with the first padlock probe, and allowing the target-binding regions of the probe to hybridise to the target nucleic acid molecule; and
(b) after the probe has hybridised to the target nucleic acid molecule, contacting the hybridised padlock probe/target nucleic acid reaction mixture with a polymerase.

4. The method of any one of claims 1 to 3, wherein the method is for detecting a variant target nucleic acid sequence in a target nucleic acid molecule in a sample, and steps (iv) to (vi) comprise:
(iv) contacting the first RCP with two or more second padlock probes each comprising target binding regions specific for different variants of the target nucleic acid sequence and allowing the probes to hybridise to their respective variant target sequence in the multiple repeats, where it is present;
(v) ligating the second padlock probes which have hybridised to circularise the hybridised padlock probes;
(vi) performing second RCA reactions using the circularised padlock probes as a second RCA template to generate a second RCP containing multiple repeat complementary copies of the second padlock probe;
(vii) detecting the second RCP to identify the second padlock probe, and thereby the variant target nucleic acid sequence.

5. The method of claim 2 or claim 4, wherein steps (i) and (ii) are cyclically repeated to generate the first RCA template.

6. The method of any one of claims 1 to 5, wherein the target nucleic acid molecule is genomic DNA.

7. The method of any one of claims 1 to 5, wherein the target nucleic acid molecule is RNA, and the method comprises generating a cDNA copy of the target RNA before contacting with the padlock probe in step (i).

8. The method of any one of claims 4 to 7, wherein the variant target nucleic acid sequence is a mutant target nucleic acid sequence or a wild-type sequence that may be present at a given position in a target nucleic acid molecule, or an allelic variant at a target position in a target nucleic acid molecule, or a polymorphism that may be present in a target nucleic acid molecule.

9. The method of any one of claim 1 to 6, or 8, wherein the target nucleic acid molecules are cell-free DNA molecules.

10. The method of any one of claims 1 to 9, wherein the sample is a liquid biopsy sample.

11. The method of claim 9 or claim 10, wherein the sample is plasma.

12. The method of any one of claims 1 to 8, wherein the variant nucleic acid sequence is detected in situ in a cell or tissue sample.

13. The method of any one of claims 1 to 12, wherein the sample is, or is prepared from, a clinical sample.

14. The method of any one of claims 1 to 13, wherein a crowding reagent is added to the target nucleic acid molecule prior to or in step (i).

15. The method of any one of claims 1 to 14, wherein in step (i) the target nucleic acid molecule is incubated with the padlock probe and other reagents at an annealing temperature of 50-65° C. for initial hybridisation of the probe, preferably at 53 to 60° C., more preferably at 55 to 60° C.

16. The method of claim 15, wherein after the initial annealing step, the temperature is reduced, for extension of a hybridised 3′ end of the probe.

17. The method of any one of claims 1 to 16, wherein in step (i) the polymerase is the Stoffel fragment of Taq polymerase and the dNTPs are provided at a concentration of no more than 1 μM, preferably nor more than 0.5 μM, and more preferably no more than 0.3, or 0.25 μM.

18. The method of any one of claims 1 to 16, wherein in step (i) the polymerase is Phusion polymerase and the dNTPs are provided at a concentration of no more than 10 μM, preferably nor more than 3 μM, and more preferably no more than 1 μM.

19. The method of any one of claims 1 to 18, wherein the target binding regions of the first padlock probe hybridise to the target nucleic acid molecule with a gap of at least 4, preferably at least 6, nucleotides in between.

20. The method of any one of claims 1 to 19, wherein the variant target nucleic acid sequence to be detected comprises a single variant base, and the variant base is not located at the position corresponding to the first or the last base of the gap between the hybridised ends of the first padlock probe.

21. The method of any one of claims 1 to 20, wherein the second padlock probes each comprises a detection sequence which is specific to the padlock probe and the second RCPs are detected by detection probes which hybridise to the complementary copies in the second RCP of the detection sequence.

22. The method of claim 21, wherein the detection probes are labelled with detectable labels, preferably with a fluorescent label.

23. The method of any one of claims 1 to 22, wherein the second RCPs are detected by microscopy or by flow cytometry.

24. The method of any one of claims 1 to 23, wherein down to step (vi) the method is a homogenous method performed in solution or suspension.

25. The method of any one of claims 1 to 24, wherein the method is performed on a solid support.

26. The method of any one of claims 1 to 25, wherein the second RCPs are detected by imaging.

27. The method of any one of claims 1 to 26, wherein the method is performed in multiplex, wherein in step (i) the sample is contacted with multiple different first padlock probes each specific for a different target nucleic acid molecule or for different target nucleic acid sequence.

28. A kit for use in detecting a target nucleic acid sequence in a target nucleic acid molecule, said kit comprising:
(i) a first padlock probe which comprises at or near its respective 5′ and 3′ ends target-binding regions which are capable of hybridising to complementary binding sites in the target nucleic acid molecule which flank the target nucleic acid sequence, and allowing the target binding regions of the probe to hybridise to the target nucleic acid molecule;
(ii) a second padlock probe which comprises target-binding regions which are specific for the target nucleic acid sequence.


SEQUENCE LISTING (5 sequences)

SEQ ID NO: 1 - DNA, 30 bp, Artificial Sequence
  Gap-fill probe - 5′ arm (5′ target binding region)
gtaggcaagagtgccttgacgatacagcta

SEQ ID NO: 2 - DNA, 53 bp, Artificial Sequence
  Gap-fill probe - non target complementary region (backbone region)
tgtatctgacttagcttccatcttcatagagcgatatcctacggtagtgtatc

SEQ ID NO: 3 - DNA, 15 bp, Artificial Sequence
  Gap-fill probe - 3′ arm (3′ target binding region)
gtggtagttggagct

SEQ ID NO: 4 - DNA, 84 bp, Artificial Sequence
  Target sequence
cctgctgaaaatgactgaatataaacttgtggtagttggagctggtggcgtaggcaagagtgccttgacgatacagctaattca

SEQ ID NO: 5 - DNA, 18 bp, Artificial Sequence
  Sequence in target molecule comprising probe binding sites and target sequences
ggagctggtggcgtaggc